(19) United States
(12) Patent Application Publication
(10) Pub. No.: US 2012/0040370 A1
(43) Pub. Date: Feb. 16, 2012

(54) SYSTEMS AND METHODS FOR RAPIDLY CHANGING THE SOLUTION ENVIRONMENT AROUND SENSORS

(75) Inventors: Owe Orwar, Paris (FR); Daniel Chiu, Seattle, WA (US); Mattias Karlsson, Onsala (SE); Johan Pihl, Olofstorp (SE); Jon Sinclair, Stockholm (SE); Frederik Pettersson, Vastra Frolunda (SE)

(73) Assignee: CELLECTRICON AB, Goteborg (SE)

(21) Appl. No.: 11/722,860

(22) PCT Filed: Oct. 25, 2006
(86) PCT No.: PCT/IB2006/004236

§ 371 Date: Sep. 8, 2011

(51) Int. Cl. C12Q 1/02; G01N 27/00; C12M 1/34

(52) U.S. Cl. 435/7.2; 435/29; 435/287.1

(57) ABSTRACT

The invention provides microfluidic systems for altering the solution environment around a nanoscopic or microscopic object, such as a sensor, and methods for using the same. The invention also provides a system and methods for modulating, controlling, preparing, and studying receptors.

### RELATED APPLICATIONS

This application claims priority to U.S. Provisional Application Ser. No. 60/356,377, filed Feb. 12, 2002; U.S. Provisional Application Ser. No. 60/404,886, filed Aug. 21, 2002; U.S. application Ser. No. 10/345,107, filed Jan. 15, 2003; and to U.S. application Ser. No. 11/031,513, filed Jan. 6, 2005; and to U.S. application Ser. No. 10/645,834, filed Aug. 20, 2003, the entire contents of each are hereby incorporated reference in their entirety.

### BACKGROUND

Of mammalian tissues, the central nervous system is one of the most complex, both in terms of structure and function. The brain has an incredible capacity for executing a multitude of computational tasks and possesses several intricate forms of memory mechanisms. Understanding the function of a variety of CNS processes in the healthy and diseased brain has been one of the most intensively studied by mankind but is still not well-established and understood.

Ion-channels and G-protein coupled receptors (GPCRs) are important therapeutic targets. Neuronal communication, heart function, and memory all critically rely upon the function of ligand-gated and voltage-gated ion-channels as well as GPCRs. In addition, a broad range of chronic and acute pathophysiological states in many organs such as the heart, gastrointestinal tract, and brain involve ion channels and GPCRs. Indeed, many existing drugs bind receptors directly or indirectly connected to ion-channels and GPCRs. For example, anti-psychotic drugs interact with receptors involved in dopaminergic, serotonergic, cholinergic and glutamatergic neurotransmission.

Because of the importance of ion-channels and GPCRs as drug targets, there is a need for methods that enable high throughput screening (HTS) of compounds acting on ligand-gated and voltage-gated channels and GPCRs (see e.g., Sinclair et al., 2002, Anal. Chem. 74: 6133-6138). However, existing HTS drug discovery systems targeting ion channels generally miss significant drug activity because they employ indirect methods, such as raw binding assays. Although as many as ten thousand drug leads can be identified from a screen of a million compounds, identification of false positives and false negatives can still result in a potential highly therapeutic blockbuster drug being ignored, and in unnecessary and costly investments in false drug leads. Patch clamp methods are superior to any other technology for measuring ion channel activity in cells, and can measure currents across cell membranes in ranges as low as picoAmps (see, e.g., Neher and Sakmann, 1976, Nature 260: 799-802; Hamill, et al., 1981, Pflugers Arch 391: 85-100; Sakmann and Neher, 1983, In Single-Channel Recording pp. 37-52, Eds. B. Sakmann and E. Neher. New York and London, Plenum Press, 1983). However, patch clamp methods generally have not been the methods of choice for developing HTS platforms. While fluorescence readout for GPCRs activity is well established, these methods lack the ability to introduce test compounds onto cells in a controlled, rapid, and parallel fashion.

### SUMMARY

The invention provides microfluidic systems for altering the solution environment around a nanoscopic or microscopic object, such as a sensor, and methods for using the same. This invention describes methods by which compounds can be applied to cells and washed from cells in a rapid and controlled fashion for HTS screening of ion channels and GPCRs in particular, and for other biological assays in general. The invention can be applied in any sensor technology in which the sensing element needs to be exposed (then subsequently unexposed) rapidly, sequentially, and controllably, to a number of different solution environments (e.g., two or greater) whose characteristics may be known or unknown. In contrast to prior art microfluidic systems, the interval between sample deliveries is minimized, e.g., on the order of microseconds and seconds, permitting rapid analysis of compounds (e.g., drugs).

According to one aspect, the invention provides a system for modulating, controlling, preparing, or studying receptors. The system comprises a substrate for changing a solution environment around a sensor, the substrate comprising a plurality of channels, each channel comprising an outlet; and a mechanism for selectively exposing a sensor to a fluid stream from an outlet, wherein each of the channels delivers a fluid stream into a chamber.

According to another aspect, the invention provides, a system for modulating, controlling, preparing, or studying receptors, comprising a chamber (e.g., open or closed) for receiving a sensor; and a plurality of channels, each channel comprising an outlet for delivering a substantially separate fluid stream into the chamber, wherein each of the channels delivers a fluid stream into the chamber.

According to yet another aspect, the invention provides a system for modulating, controlling, preparing, or studying receptors, comprising a substrate for changing a solution environment around a sensor, the substrate comprising a plurality of channels, each channel comprising an outlet for delivering a substantially separate fluid stream to a sensor; and a processor for controlling delivery of fluid from each channel to the sensor, wherein each of the channels delivers a fluid stream into the chamber.

In one aspect, at least one channel is in communication with a reservoir. In a related aspect, a system has a plurality of buffer reservoirs and sample reservoirs. In another related aspect, each reservoir is in communication with a different channel. In yet another related aspect, the system has alternating sample and buffer reservoirs.

In another aspect, the system further comprises a mechanism for applying positive or negative pressure to the reservoir.

In one aspect, the scanning mechanism comprises a mechanism for varying pressure across one or more channels.

In another aspect, the system further comprises at least one drain channel communicating with the chamber.

According to one aspect, the system further comprises a mechanism for holding a sensor, which is coupled or connected to a positioner for positioning the sensor in proximity to an outlet of a channel. In a related aspect, the mechanism for holding the sensor comprises a mechanism for holding a cell. In another related aspect, the sensor comprises a cell or a portion of a cell. Another related aspect provides, a cell as a patch clamped cell or patch-clamped cell membrane fraction. Yet another related aspect provides, a cell or portion of the cell comprises an ion channel. Another related aspect provides a cell attached to an opening that is smaller than the cell. Still another related aspect provides a cell or portion of a cell is selected from cultured cell, a bacterial cell, a protist cell, a yeast cell, a plant cell, an insect cell, an avian cell, an amphibian cell, a fish cell, a mammalian cell, an oocyte, a cell expressing a recombinant nucleic acid, and a cell from a patient with a pathological condition.

According to one aspect, the cell or portion of the cell is positioned in proximity to the outlet of a channel using a positioner.

In one aspect, the system further comprises a sensor selected from a surface plasmon energy sensor; a fluorescence sensor, an FET sensor, an ISFET; an electrochemical sensor, an optical sensor; an acoustic wave biosensor; a sensor comprising a sensing element associated with a Quantum Dot particle; a polymer-based biosensor; and an array of biomolecules immobilized on a substrate. In a related aspect, wherein the system comprises a plurality of sensors.

In another aspect, the system further comprises a mechanism for varying pressure across one or more channels in the substrate for selectively exposing a cell to a fluid stream.

In another aspect, the system further comprises an exposing mechanism for selectively exposing a sensor to a fluid stream by varying the laminar steam via pressure that the cell is exposed to. In related aspect, the exposing mechanism comprises a mechanism for varying pressure across one or more channels in the substrate sequentially.

In another aspect, the system further comprises a processor in communication with the exposing mechanism. In a related aspect, the processor controls one or more of: the rate of exposing, the direction of scanning, acceleration of scanning, number of scans, pause intervals at a channel and pressure changes across one or more channels. In another related aspect, the processor controls the exposure time. In another aspect, the system further comprises a detector in communication with the sensor for detecting the responses of a sensor in the chamber. In a related aspect, the detector communicates with a processor comprising a data acquisition system.

In one aspect, the system is interfaced to a fluid delivery system operably linked to a micropump for pumping fluids from the fluid delivery system into one or more reservoirs or channels of the substrate. In a related aspect, the fluid delivery system is capable of sequentially delivering different types of samples and/or buffer to the sensor chamber or channel or reservoirs of the substrate. In another related aspect, the fluid delivery system is capable of programmably delivering a selected volume or concentration of sample or buffer to at least one reservoir or sample chamber or channel. In yet another related aspect, the system has alternating sample and buffer reservoirs. In another related aspect, the fluid delivery system is capable of programmably delivering a selected volume or concentration of sample or buffer to at least one reservoir, sensor chamber or channel at a selected time interval.

In another aspect, the system further comprises at least one output or waste channel or reservoir for removing fluid from the system.

In another aspect, the system further comprises a mechanism for delivering positive or negative pressure to at least one of the channels or a reservoir. In a related aspect, the mechanism for delivering pressure is in communication with a processor.

In one aspect, the substrate comprises a material selected from a crystalline semiconductor material; silicon; silicon nitride; Ge, GaAs; metals; Al, Ni; glass; quartz; crystalline insulator; ceramics; plastics; an elastomeric material; silicone; EPDM; Hostaflon; a polymer; a fluoropolymer, Teflon®; polymethylmethacrylate; polydimethylsiloxane; polyethylene; polypropylene; polybutylene; polymethylpentene; polystyrene; polyurethane; polyvinyl chloride; polyarylate; polyarylsulfone; polycaprolactone; polyestercarbonate; polyimide; polyketone; polyphenylsulfone; polyphthalamide; polysulfone; polyamide; polyester, epoxy polymer; thermoplastic; an organic material; an inorganic material; combinations thereof.

In one aspect, the substrate is three-dimensional and at least two of the channels lie at least partially in different planes.

In one aspect, provided herein are substrates comprising an open-volume chamber for the sensor, and a plurality of channels. A plurality, if not all, of the channels programmably deliver a fluid stream into the sensor chamber.

In a preferred aspect, each channel of the substrate comprises at least one inlet for receiving solution from a reservoir, conforming in geometry and placement on the substrate to the geometry and placement of wells in a multi-well plate. For example, the substrate can comprise 96-1024 reservoirs, each connected to an independent channel on the substrate. Preferably, the center-to-center distance of each reservoir corresponds to the center-to-center distance of wells in an industry standard microliter or multi-well plate.

In a further aspect, the substrate comprises one or more treatment chambers or microchambers or channel for delivering a treatment to a cell or cells placed within the treatment chamber or channel (also referred to herein as a sensor chamber or channel). The treatment can comprise exposing the cell to a chemical or compound, (e.g. drugs or dyes, such as calcium ion chelating fluorogenic dyes), exposing the cell to an electrical current (e.g., electroporation, electrofusion, and the like), or exposing the cell to light (e.g., exposure to a particular wavelength of light). A treatment chamber or channel can be used for multiple types of treatments which may be delivered sequentially or simultaneously. For example, an electrically treated cell also can be exposed to a chemical or compound and/or exposed to light. Treatment can be continuous over a period of time or intermittent (e.g., spaced over regular or irregular intervals). The cell treatment chamber can comprise a channel with an outlet for delivering a treated cell to the sensor chamber or directly to a mechanism for holding the cell connected to a positioner (e.g., a micropositioner or nanopositioner) for positioning the cell within the chamber.

Preferably, the base of the sensor chamber is optically transmissive and in one aspect, the system further comprises a light source (e.g., such as a laser) in optical communication with the sensor chamber or channel. The light source can be used to continuously or intermittently expose the sensor to light of the same or different wavelengths. The sensor chamber and/or channels additionally can be equipped with control devices. For example, the sensor chamber and/or channels can comprise temperature sensors, pH sensors, and the like, for providing signals relating to chamber and/or channel conditions to a system processor.

The sensor chamber or channel can be adapted for receiving a variety of different sensors. In one aspect, the sensor comprises a cell or a portion of a cell (e.g., a cell membrane fraction). In another aspect, the cell or cell membrane fraction comprises an ion channel, including, but not limited to, a presynaptically-expressed ion channel, a ligand-gated channel, a voltage-gated channel, and the like. In a further aspect, the cell comprises a receptor, such as a G-Protein-Coupled Receptor (GPCR), or an orphan receptor for which no ligand is known, or a receptor comprising a known ligand.

A cultured cell can be used as a sensor and can be selected from, for example, CHO cells, NIH-3T3 cells, and HEK-293 cells, and can be recombinantly engineered to express a sensing molecule such as an ion channel or receptor. Many other different cell types also can be used, which can be selected from, for example, mammalian cells (e.g., including, but not limited to human cells, primate cells, bovine cells, swine cells, other domestic animals, and the like); bacterial cells; protist cells; yeast cells; plant cells; invertebrate cells, including insect cells; amphibian cells; avian cells; fish; and the like.

A cell membrane fraction can be isolated from any of the cells described above, or can be generated by aggregating a liposome or other lipid-based particle with a sensing molecule, such as an ion channel or receptor, using methods routine in the art.

The cell or portion of the cell can be positioned in the chamber using a mechanism for holding the cell or cell portion, such as an opening of a channel or a pipette (e.g., a patch clamp pipette) or a capillary connected to a positioner (e.g., such as a micropositioner or nanopositioner or micromanipulator), or an optical tweezer.

In one aspect, the base of the chamber or channel comprises one or more openings and the cell or portion of the cell is placed at the opening which can be in communication with one or more electrodes (e.g., the sensor can be integral with a planar patch clamp chip).

Non-cell-based sensors also can be used in the system. Suitable non-cell based sensors include, but are not limited to: a surface plasmon energy sensor; an FET sensor; an ISFET; an electrochemical sensor; an optical sensor; an acoustic wave sensor, a sensor comprising a sensing element associated with a Quantum Dot particle; a polymer-based sensor; a single molecule or an array of molecules (e.g., nucleic acids, peptides, polypeptides, small molecules, and the like) immobilized on a substrate. The sensor chamber also can comprise a plurality of different types of sensors, non-cell based and/or cell-based. However, an object placed within a chamber need not be a sensor. For example, the object can be a colloidal particle, beads, nanotube, a non-sensing molecule, silicon wafer, or other small elements.

The system can be used to rapidly, programmably, and sequentially, change the solution environment around a cell which has been electroporated and/or electrofused, and/or otherwise treated within the cell treatment chamber or channel. Alternatively, or additionally, the sensor chamber or channel also can be used as a treatment chamber and in one aspect, the sensor chamber or channel is in electrical communication with one or more electrodes for continuously or intermittently exposing a sensor to an electric field.

Provided herein, according to one aspect, are methods for modulating, controlling, preparing, or studying receptors, comprising providing a substrate, the substrate comprising: a sensor channel comprising a plurality of sensor positioning channels, and a delivery channel configured to deliver one or more of an agent, agonist, or antagonist to the sensor chamber; and sequentially exposing a biosensor to different fluid streams optionally comprising an agent, wherein sensors are associated with one or more sensor positioning channels.

In one embodiment, the sensor is a cell or a vesicle.

In another embodiment, the sensor positioning channel is a patch channel.

In one embodiment, sequentially exposing comprises solution exchange around a sensor.

In one embodiment, the solution exchange is pressure driven.

In another embodiment, the substrate further comprises one or more of at least one pressure source adapted to provide positive and negative pressure to the sensor channel and the plurality of sensor positioning channels; a buffer reservoir in fluid communication to the sensor channel; an inlet reservoir in fluid communication with the sensor channel; an inlet reservoir in fluid communication with the sensor channel; or a waste reservoir in fluid communication with the sensor channel.

In one embodiment, the methods further comprise applying negative pressure from the waste reservoir.

In one embodiment, the methods further comprise applying positive pressure to the buffer reservoir.

In one embodiment, the methods further comprise applying negative pressure on the plurality of sensor channels.

In one embodiment, the openings of the sensor positioning channels comprise protruded surfaces.

In one embodiment, the protruded surface defining an opening comprises one or more of a microchannel, a column, a pyramidal element, rod or reeve.

In another embodiment, electrical resistance between a sensor and the system comprises at least about 100 Mohm.

In another embodiment, the method further comprises measuring electrical properties of the cell.

In one embodiment, the sensor chamber comprises a buffer, at least one agonist, at least one antagonist, at least one sample, or a combination thereof.

In one embodiment, the exposing is selectively exposing the biosensor to a selected concentration of sample.

In another embodiment, the exposing is selectively for a selected time.

In one embodiment, the methods further comprise providing to the sensor positioning channels one or more buffers.

In one embodiment, the methods further comprise exposing the biosensor to the one or more buffers.

In one embodiment, the exposing the biosensor to one or more buffers is interspersed between the exposing to one or more samples.

In another embodiment, the exposing to one or more buffers is a wash period.

In one embodiment, the exposing to one or more buffers is a rest period.

In another embodiment, the exposing to one or more buffers is a wash and a rest period.

In another embodiment, a rest period in buffer is between a series of sample exposures and interdigitated by one or more wash periods in buffer.

In another embodiment, the receptors are exposed to ligand solutions in order of increasing concentrations

In one embodiment, the receptors are exposed to ligand solutions in order of decreasing concentrations

In another embodiment, the agent is a candidate drug; a known drug; a suspected carcinogen; a known carcinogen; a candidate toxic agent, a known toxic agent; and an agent that acts directly or indirectly on ion channels.

In another embodiment, the method is method for studying the memory properties of a receptor.

In one embodiment, the memory functions are short-term, medium-term, or long-term memory functions.

In another embodiment, effects of an agent on memory properties of a biosensor are studied.

In one embodiment, the exposing further comprises producing pressure drops across one or more channels.

In another embodiment, the cell-based biosensor comprises a patch-clamped cell or patch-clamped cell membrane fraction.

In one embodiment, the cell-based biosensor comprises an ion-channel.

In one embodiment, the ion-channel is a G-Protein Coupled Receptor.

Provided herein, according to one aspect, are systems comprising a substantially planar substrate in communication with at least one conducting element, wherein the substantially planar structure comprises a sensor channel comprising a plurality of sensor positioning channels.

In one embodiment, the sensor positioning channels comprise electrode channels.

In one embodiment, the systems further comprise at least one pressure source adapted to provide positive and negative pressure to the sensor channel and the plurality of sensor positioning channels.

In one embodiment, the systems further comprise a buffer reservoir in fluid communication to the sensor channel.

In one embodiment, the systems further comprise an inlet reservoir in fluid communication with the sensor channel.

In one embodiment, the systems further comprise a waste reservoir in fluid communication with the sensor channel.

In one embodiment, the systems further comprise a mechanism for providing fluid flow for establishing and maintaining an electrically resistant seal between a cell and a conducting element.

In one embodiment, the openings of the sensor positioning channels comprise protruded surfaces.

In another embodiment, the protruded surface defining an opening comprises one or more of a microchannel, a column, a pyramidal element, rod or reeve.

In one embodiment, electrical resistance between a sensor and the system comprises at least about 100 Mohm.

In one embodiment, the system is used for one or more of patch clamping measuring a parameter of a sensor.

In another embodiment, the parameter measured using fluorescence.

In one embodiment, the parameter is one of more of an ion channel activity, such as, for example, currents across sensor membranes, voltage across the membranes, or capacitance across the membranes.

Provided herein, according to one aspect, are systems for rapid switching, comprising a substantially planar substrate in communication with at least one conducting element, wherein the substantially planar structure comprises: a sensor chamber comprising a plurality of sensor positioning channels, a delivery channel, at least one buffer/agent delivery channel in communication with the sensor chamber, a waste channel in communication with the sensor chamber, a buffer well, a negative pressure source communicated through the waste channel, and a switching pressure source communicated through the buffer well, and a ground electrode.

In another embodiment, wherein the buffer/agent delivery channels are from between about 25 to about 45 um wide and about 15 to about 45 um high and converge to a single channel that is from between about 55 to about 85 um wide.

In one embodiment, the buffer/agent delivery channels are about 35 um wide and about 30 um high and converge to a single channel that is about 70 um wide.

In one embodiment, the sensor chamber is from between about 50 to about 100 um wide and from between about 15 to about 45 um high.

In another embodiment, the sensor chamber is about 70 um wide and about 30 um high.

In one embodiment, the delivery channel is from between about 50 to about 100 um wide and from between about 15 to about 45 um high. In another embodiment, the delivery channel is bout 70 um wide and about 30 um high.

In one embodiment, the sensor positioning channels comprise openings into the sensor chamber and wherein the opening are from between about 50 um long.

In one embodiment, the sensor positioning channels after between about a 25 to about a 75 um section widens to between about 25 to about 75 um wide and between about 15 to about 45 um high.

In another embodiment, the sensor positioning channels after about a 50 um section widen to between about 50 um wide and about 30 um high.

In one embodiment, the buffer well comprises a volume of between about 5 uL and about 30 uL.

In one embodiment, the systems further comprises a waste well in communication with the waste channel.

In one embodiment, the waste well comprises a volume of between about 5 uL and about 30 uL.

In another embodiment, the conducting element comprise electrodes.

In one embodiment, the ground electrode is contained within the waste chamber.

In another embodiment, the sensor positioning channels are in communication with wells for communicating pressure.

In one embodiment, the sensor positioning channels are the same length, wherein the sensor positioning channels comprise electrodes.

Provided herein, according to one aspect, are methods for modulating, controlling, preparing, or studying receptors, comprising providing a microfluidic system, wherein the microfluidic system comprises a substrate in communication with at least one conducting element, wherein the substantially planar structure comprises: a sensor chamber comprising a plurality of sensor positioning channels, a delivery channel, at least one buffer/agent delivery channel in communication with the sensor chamber, a waste channel in communication with the sensor chamber, a buffer well, a negative pressure source communicated through the waste channel, and a switching pressure source communicated through the buffer well, and a ground electrode; capturing a biosensor at an opening of a sensor positioning channel; and exposing the biosensor to an agent.

In one embodiment, the methods further comprise exposing the biosensor to a buffer, wherein the switching between buffer and agent is rapid.

In one embodiment, rapid comprises between about 10 μs and about 100 seconds.

In another embodiment, switching between buffer and agent comprises a switching pressure of between about −7.6 and about −9.6 kPa.

In one embodiment, a capture pressure is applied to the system and comprises from between about 0.4 to about 0.8 kPa.

In one embodiment, a driving pressure is applied to the system after a biosensor is captured and comprises from between about −7 to about −9 kPa.

In another embodiment, switching between buffer and agent is done one or more times.

In one embodiment, switching between buffer and agent is done at a rate of five time in about 4.5 seconds.

In another embodiment, switching between buffer and agent comprises a fluidic switch time.

In another embodiment, the fluidic switch time comprises from between about 15 to about 35 ms.

Thus, the system can, for example, be used to characterize if an ion channel or receptor antagonists is a competitive or non-competitive inhibitor. The systems and methods according to the invention also can be used for toxicology screens, e.g., by monitoring cell viability in response to varying kinds or doses of compound, or in diagnostic screens. The method can also be used to internalize drugs, in the cell cytoplasm, for example, using electroporation to see if a drug effect is from interaction with a cell membrane bound outer surface receptor or target or through an intracellular receptor or target. It should be obvious to those of skill in the art that the systems according to the invention can be used in any method in which an object would benefit from a change in solution environment, and that such methods are encompassed within the scope of the instant invention.

Other aspects and embodiments are describe infra.

### BRIEF DESCRIPTION OF THE FIGURES

The objects and features of the invention can be better understood with reference to the following detailed description and accompanying drawings. The Figures are not to scale.

FIG. 1 shows a 3-D perspective illustration of the reservoirs comprising a 6-patch site microfluidic patch clamp chip. −p1-Negative pressure source controlling cell delivery and compound perfusion. −p2-Negative pressure source controlling cell immobilization; +p3-Positive pressure source controlling rinsing buffer flow. Patch clamp electrodes (AgCl) are placed in the patch clamp channel wells, one common ground electrode is placed in the waste well. Interfacing was achieved in the same way as in the Dynaflow chips (plastic lid attached with double adhesive tape

FIG. 2A shows a microfluidic patch-clamp chip incorporating 4 multiplexed 6-patch site units. FIG. 2B shows a single 6-patch site unit isolated and magnified from the 4-unit multiplex chip of FIG. 2A. FIG. 2C is a schematic showing the arrangement of the inflow channels and the patch channels fluidly connected with the delivery channel 100; magnified from the corresponding area outlined in FIG. 2B. FIG. 2D is a schematic further magnifying and isolating the arrangement of the six patch sites 150 in relation to the delivery channel 100.

FIG. 3 depicts a schematic drawing showing the functional components of the microfluidic six-patch site unit.

FIG. 4 depicts a step in the operation of a six-patch site unit, including a step of filling the chip with buffer solution.

FIG. 5 depicts a step in the operation of a six-patch site unit, including a step of adding cells to the inlet reservoir, transport to patch channels by negative pressure pumping from waste well (−p2). Immobilize cells in patch channel by negative pressure (−p1).

FIG. 6 depicts a step in the operation of a six-patch site unit, including a step of applying positive pressure on buffer well (+p), creates protective sheat flow over the patched cells, continue to apply negative pressure −p2 to empy inlet well.

FIG. 7 depicts a step in the operation of a six-patch site unit, including a step of adding a substance to inlet well, and pumping the substance towards waste well by −p2 cells are protected by sheat flow from buffer well.

FIG. 8 depict another step in the operation of a six-patch site unit, including a step of removing positive pressure on buffer well (+p), sheat flow is terminated, cells are exposed to compound.

FIG. 9 depicts another step in the operation of a six-patch site unit, including the step of Sapplying positive pressure on buffer well (+p), protective sheat flow is created, new concentration of compound can be added to the inlet well and the cycle can be repeated.

FIG. 10 depicts the a flow chart of the circuitry associated with one side of a six-patch site unit.

FIG. 11 depicts rapid solution switch on ligand gated ion channels. The recordings were performed on WSS-1 cells expressing GABAA ion channel, immobilized and patch-clamped in a Nanoflow unit cell. The agonist; 500 μM GABA was applied between approximately 1.3 s and 4 s. Before and after GABA application, the cells were rinsed with extracellular buffer. Data acquisition was performed by HEKA EPC10 triple amplifier. The data indicates an average full solution exchange time of 55 ms for the three cells when switching on the agonist.

FIG. 12 depicts a schematic a microsystem for cell capturing and patch-clamp measurement in a closed sensor chamber. P1-6 are wells with patch-clamp Ag/AgCl electrodes and pressure connections to capture and hold cells. W is waste well with patch-clamp counter electrode and pressure connection for driving pressure. O is an open well for different solutions and S is the Buffer/Switching well with pressure connection for the switching pressure source. B is a closeup of the channel configuration at the sensor chamber. Green is 2 μm high patch-channels, blue is 30 μm high fluidic channels and red is wells extending through the entire microfluidic chip.

FIG. 13 depicts a microscope image of sensor chamber and channels in microfluidic PDMS chip.

FIG. 14 depicts a microfluidic switching captured by fluorescence microscopy using fluorescein. A low level of brightfield illumination is also used to show the outlines of the microchannels. There are cells captured at all openings in the sensor chamber and in this case the cells were quite sticky, resulting in groups of many cells captured at some openings. A. When the switch/buffer well is connected to atmospheric pressure the cells are protected by a sheath flow of buffer. B. when a negative pressure balancing the waste pressure is applied the solution from the open well reaches the cells. C. The fluorescence level at three patch-sites where PS1 is closest to the chamber inflow and PS3 is furthest downstreams. The fluorescence levels are nonmed against min and max values for each site. The fluidic system is switched rapidly five times in 4.5 s. This data is acquired through time-lapse capture of frames at 40 ms interval, which are then subject to ROI analysis with ROIs defined as a half-moon shaped polygon on the outside of the cell-membranes visible in A and B.

FIG. 15 depicts a patch-clamp current response from GABAA ion channels activated with 1 mM GABA. A shows the entire GABA response, including the following buffer rinse and B shows the risetime of the signal, indicating a fluidic switch time in the 25 ms range.

### DETAILED DESCRIPTION

Provided herein is a system and method for rapidly and programmably altering the local solution environment around a sensor, such as a cell-based biosensor. The invention relates to methods of determining novel functions in receptor proteins, situated, e.g., in the central nervous system and other cellular systems. The method is based on a microfluidic protocol to expose a cell or other preparation containing the receptor-protein to ligands with precise control of periods of exposure to ligand, ligand concentration, wash times between ligand exposure, and order of application. In particular, a protocol for cyclic scanning patch clamp where a patch-clamped cell is exposed periodically to ascending and descending concentrations of ligands with controlled exposure times is demonstrated. The methods can additionally be used for characterization and validation of receptor modulators such as drugs and pharmaceutically active substances.

The following definitions are provided for specific terms which are used in the following written description.

As used in the specification and claims, the singular form “a”, “an” and “the” include plural references unless the context clearly dictates otherwise. For example, the term “an opening” includes a plurality of openings. The term “an ion-channel” includes a plurality of ion channels. The term “an opening” or “the opening” can refer to a plurality of openings.

As used herein, a “patch clamp device” is a device suitable for obtaining patch clamp recordings. Such a device comprises, for example, an insulating surface for separating a cell membrane from an electrode. The surface comprises an opening that couples the cell to the electrode through an electrolyte solution in a lumen or column defining the opening, such that the cell is in electrical communication with the electrode (e.g., exposed to an electrical field created by the electrode and capable of transmitting an electrical signal, such as a current or voltage, back to the electrode). The insulating surface may be, for example, fabricated in the form of a wafer or chip comprising a plurality of sensor chambers, wells, or columns, each for receiving a cell (e.g., the opening of a channel may receive a cell). The base of a sensor chamber or a column may comprise an electrical contact zone associated with one or more electrodes, while the opening of the chamber or the column receives the cell. The sensor chamber or column (patch site), like the micropipette of a traditional patch clamp device, is. filled with an electrolyte solution for coupling the cell to an electrode, such that the cell is in communication with the electrode(s).

A “cell holding,” “sensor holding,” “sensor positioning,” “sensor measurement” or a “cell measurement” device is a device suitable for obtaining various measurements or recordings. Such a device comprises, for example, a surface for holding a cell in place. The surface comprises, for example, an opening to a channel or chamber. The surface may be, for example, fabricated in the form of a wafer or chip comprising a plurality of sensor chambers, wells, or columns, each for receiving a cell (e.g., the opening of a channel may receive a cell).

As used herein, the term “electrode” refers to a device that transmits or conducts electric signals.

As used herein, the term “electrolyte solution”, refers to the solution within a sensor chamber of a patch clamp array device or within a micropipette.

As used herein, the term “bath solution” refers to the solution or medium surrounding the cell outside of a sensor chamber or column or outside of a patch clamp micropipette. Preferably, a bath solution used for measuring the ionic current through a biological membrane of a cell may be chosen so that it is similar to the external ionic environment that the cell is exposed to in vivo.

As used herein, the term “opening” refers to any aperture or orifice, such as a hole, gap or slit. The opening can take any shape or form; for example, it may be substantially elliptical, circular, square, or polyhedral. Openings used in patch clamp or cell or sensor holding or measurement systems described herein range in size from about 0.1 micron to about 100 microns. However openings can range from at least about 0.01 μm, at least about 0.05 μm, at least about 0.1 μm, at least about 10 μm, at least about 15 μm, at least about 20 μm, at least about 50 μm, at least about 75 μm, or at least about 100 μm.

As used herein a “surface defining an opening” refers to a surface which includes an opening and which couples a cell to an electrode compartment or channel or a cell holding or measurement chamber or channel. Typically, a surface defining an opening refers to that portion of the surface in contact with a cell membrane (e.g., such as the rim of a micropipette and the inner surface of the micropipette tip which contacts the cell membrane as a seal is formed, or in the case of an on-chip device, the rim of a sensor chamber and the portions of the walls of the sensor chamber which contact the cell when it is sealed against the sensor chamber or an opening of a channel).

As used herein, “an electrode compartment or channel” refers to one or more electrodes and lumen or column comprising an electrolyte solution which couples the one or more electrodes to a surface with an opening, to enable it to generate an electrical field at the opening or to receive electrical signals, such as current or voltage, for recording. The systems described herein may have one or more electrode compartment or channels and one or more electrodes that may be individually controllable. As used herein, a “sensor holding or measurement compartment or channel” refers to one or more compartments or channels having openings, for example, into a sensor chamber. The compartment or channel may be comprise buffer or other solution. The solution may, for example, contain nutrients, buffers, drugs, drug candidates, and the like.

As used herein, a “sensor chamber” generally refers to a chamber, well, column, channel, depression or reservoir in a substrate for receiving one or more cells. In the context of a cell-based biosensor adapted a patch clamp device, a sensor chamber is a chamber for receiving and positioning a cell in proximity to a patch clamp column. The chamber may comprise bath solution. In the context of an on-chip patch clamp device, the chamber may receive a single cell or multiple cells. In the context of an on-chip fluorescence measurement, the chamber may be a large channel where the cells are immobilized. For example from about 1 to about 20 or more cells. The chamber may also comprise one or more electrodes. The chamber is preferably designed in a way that restricts the motion of a cell received in the chamber, and in the case of patch-clamp measurements, comprises electrolyte solution for maintaining the cell in electrical communication with the electrodes at the base of the chamber.

As used herein, the term “cell membrane” refers to a lipid bilayer surrounding a biological compartment, and includes the membranes of natural or artificial cells (e.g., such as liposomes), membrane vesicles or portions thereof. The term “cell membrane” encompasses an entire cell comprising such a membrane, a portion of a cell, an artificial cell, or a portion of an artificial cell. Cell membrane also includes organelle membranes and portions thereof.

As used herein, a “patch” recording refers to a recording in which the patch clamp device collects ionic current passing through a membrane patch sealed against the opening of a patch clamp device. As used herein, a “whole-cell recording” refers to a set-up in which the membrane patch is ruptured, giving direct electrical access to a cell's interior.

As used herein, the term “high electrical resistance seal” refers to a seal between cell membrane and the opening of a surface separating the cell from an electrode compartment or channel, whose integrity is shown by a high electrical resistance, which is preferably, greater than about 100 MΩ, greater than about 200 MΩ, greater than about 300 MΩ, greater than about 400 MΩ, greater than about 500 MΩ, greater than about 600 MΩ, greater than about 700 MΩ, greater than above 800 MΩ, greater than about 900 MΩ, greater than about 1 GΩ, greater than about 1.2 GΩ, greater than about 1.3 GΩ, greater than about 1.4 GΩ, greater than about 1.5 GΩ, greater than about 1.6 GΩ, greater than about 1.7 Gω, greater than about 1.8 GΩ, greater than about 1.9 GΩ, greater than about 2 GΩ, greater than about 10, about 20, about 30, about 40, about 50, about 60, about 70, about 80, about 90, about 100, about 150, or 200 GΩ.

As used herein, a “microchannel” refers to a groove in a substrate comprising two walls, a base, at least one inlet and at least one outlet. In one aspect, a microchannel also has a roof. The term “micro” does not imply a lower limit on size, and the term “microchannel” is used herein interchangeably with “channel.” For example, a microchannel ranges in size from about 0.1 μm to about 1000 μm, or ranging from, 1 μm to about 500 μm.

As used herein, the term “substantially separate aqueous streams” refers to collimated streams with laminar flow.

As used herein, the term “receptor” refers to a macromolecule capable of specifically interacting with a ligand molecule.

As used herein, the term “in communication with” refers to the ability of a system or component of a system to receive signals or input data from another system or component of a system and to provide an output response in response to the input data. “Output” may be in the form of data or may be in the form of an action taken by the system or component of the system or a signal delivered by the system or component of the system (e.g., to a detector). For example, a cell in “electrical communication” with an electrode refers a cell which receives a signal from an electrode (such as a voltage, or current, etc) and which provides a response to the signal in the form of a measurable change in an electrical property (e.g., such as a current). Similarly, in fluorescence measurements, the cell may be in “optical communication” with a photon detector via the emission of light from the cell onto the detector. “In communication,” as well as “fluidly connected” may also refer to channels and reservoirs of the system being able to pass fluid from one to another. One of skill in the art will know from the context which definition of communication to apply.

As used herein, a “substantially planar substrate comprising a nonplanar element for establishing electrical communication with a cell” refers to substrate which comprises an element whose surface is elevated or depressed relative to the surface of a substrate. For example, a “non-planar element” may be pyramidal shaped, toroidal shaped, comprise a plurality of stacked planar elements or the like.

As used herein, “a measurable response” refers to a response which differs significantly from background as determined using controls appropriate for a given technique.

As used herein, a “recording” refers to collecting and/or storing data obtained from processed electrical or optical signals, such as are obtained in patch clamp or fluorescence analysis.

The methods and systems herein may be used with fluorescence-based detection techniques to study ligand binding to target proteins, such as ion channels or G-protein-coupled receptor proteins (GPCRs). This could be performed by having, for example, an ion channel of interest in, for example, a lipid bilayer membrane or a cell and a reporter probe. The probe reports changes in concentration of the ion which the channel specifically allows through when a ligand is attached to it. Ligands, which are potential drug candidates, can thus be screened using this technique, for example, through an increase in fluorescence. Upon binding, the ion channel opens and allows the ion for which it is specific to enter through the lipid bilayer membrane. The reporter probe thereby fluoresces and a signal can be observed.

As used herein, a “positioner” refers to a mechanism or instrument that is capable of holding and maintaining an object or device (e.g., a substrate, a sensor, a cell, a mechanism for holding a sensor, etc.) to which it is associated. Preferably, the positioner can also control movement of an object over distances such as nanometers (e.g., the petitioner is a nanopositioner), micrometers (e.g., the positioner is a micropositioner) and/or millimeters. Suitable positioners move, for example, in an x-, y-, or z-direction. In one aspect, positioners according to the invention also rotate about any pivot point defined by a user. In a preferred aspect, the positioner is coupled to a drive unit that communicates with a processor, allowing movement of the object to be controlled by the processor through programmed instructions, use of joysticks or other similar instruments, or a combination thereof. As used herein, “a mechanism for holding a sensor” refers to a device for receiving at least a portion of a sensor to keep the sensor in a relatively stationary position relative to the mechanism. In one aspect, the mechanism comprises an opening for receiving at least a portion of a sensor. For example, such mechanisms include, but are not limited to: a patch clamp pipette, a capillary, a column or channel, a hollow electrode, and the like.

As used herein, a “chamber” refers to an area formed by walls (which may or may not have openings) surrounding a base. A chamber may be “open volume” (e.g., uncovered) or “closed volume” (e.g., covered by a coverslip, for example). A “sensor chamber” is one which receives one or more sensors and comprises outlets in one or more walls from at least two microchannels. However, a sensor chamber according to the invention generally can receive one or more nanoscopic or microscopic objects, without limitation as to their purpose. A sensor chamber can comprise multiple walls in different, not necessarily parallel planes, or can comprise a single wall which is generally cylindrical (e.g., when the chamber is “disc-shaped”). It is not intended that the geometry of the sensor chamber be a limiting aspect of the invention. One or more of the wall(s) and/or base can be optically transmissive. Generally, a sensor chamber ranges in size but is at least about 1 μm. Sensor chambers may be channel-like in structure and be in fluid communication with buffer reservoirs and inlet reservoirs. In one aspect, the dimensions of the chamber are at least large enough to receive at least a single cell, such as a mammalian cell and may receive up to hundreds of cells.

As used herein, a “sensor” refers to a device comprising one or more molecules capable of producing a measurable response upon interacting with a condition in an aqueous environment to which the molecule is exposed (e.g., such as the presence of a compound which binds to the one or more molecules). In one aspect, the molecule(s) are immobilized on a substrate, while in another aspect, the molecule(s) are part of a cell (e.g., the sensor is a “cell-based biosensor”).

As used herein, “aqueous” is inclusive of other liquid that are not aqueous, such as alcohols, and other non-aqueous fluids and liquids.

As used herein, the term, “a cell-based biosensor” or “biosensor” refers to an intact cell or a part of an intact cell (e.g., such as a membrane patch) which is capable of providing a detectable physiological response upon sensing a condition in an aqueous environment in which the cell (or part thereof) is placed. In one aspect, a cell-based biosensor is a whole cell or part of a cell membrane in electrical communication with an electrically conductive element, such as a patch clamp electrode or an electrolyte solution. In certain embodiments, receptors and reconstituted receptor proteins within a lipid bilayer of any constitution, or similar preparations are included within the meaning of the term biosensor.

As used herein, “preparing a receptor” refers to methods of creating a receptor or receptors in a discrete kinetic state, which is characterized by having different response functions, dynamic range EC₅₀, and Hill slope. This may be done, for example, by cyclic scanning patch clamp methods described herein.

As used herein, “sequentially” is intended to encompass in sequence, in succession, consecutively and in sequences. Alternately, there may be interruption or interdigitation. As used herein, “sequentially exposing” refers to exposing a biosensor to a ligand, sample, agonist, or antagonist in sequence, in succession, consecutively, serially, or alternately, there may be interruption or interdigitation of the ligand, sample, agonist, or antagonist with buffer.

As used herein, “controller” refers to a device, for example, a programmed processor, to control or direct the methods described herein. For example, the controller may control the exposure time, sample concentration, washes, and rest time periods. The controller may also, or independently control the pressure, the position of the sensor, pulses of buffer or sample, exchange of solution on or surrounding the biosensor

As used herein, “selected time interval” or “selected length of time” refers to a time interval set to achieve a desired result or for the purpose of the studying a receptor. For example, times may be selected for the exposure time, the wash time, or the rest time.

As used herein, “sample” refers to a solution or material provided that is of interest in relation to the receptor. The sample may contain a ligand, an agonist, or antagonist or a compound or composition that is unknown and is to be studied.

As used herein, “or” may be inclusive as well as exclusive.

As used herein, “memory properties of a receptor” refers to the receptors response function or functions that are altered due to previous events (e.g., stimulations), which may be dependent on the nature of the stimuli, the wash time, and/or the magnitude (concentration) of the previous stimuli. For example, the response function to a specific concentration of agonist may be changed dependent on the history of the application of the previous stimulus.

As used herein, “short-term, medium-term, or long-term memory functions” refer to plasticity of the receptors. For example, short term includes the plasticity of the receptor lasts for between about 1 μs to about 1 s; medium term includes the plasticity that lasts for between about is to about 10 minutes; long term refers to the plasticity that lasts for between about 10 minutes to about 5 days. Plasticity refers to the receptor's ability to remember and change its response function due to previous events (e.g., stimuli).

As used herein, “periodically resensitized” or “periodically responsive” refers to an ion-channel which is maintained in a closed (e.g., ligand responsive) position when it is scanned across microchannel outlets providing samples suspected or known to comprise a ligand.

As used herein, a “substantially separate fluid stream” refers to a flowing fluid in a volume of fluid (e.g., such as within a chamber or a channel) that is physically continuous with fluid outside the stream within the volume, or other streams within the volume, but which has at least one bulk property which differs from and is in non-equilibrium from a bulk property of the fluid outside of the stream or other streams within the volume of fluid. A “bulk property” as used herein refers to the average value of a particular property of a component (e.g., such as an agent, solute, substance, or a buffer molecule) in the stream over a cross-section of the stream, taken perpendicular to the direction of flow of the stream. A “property” can be a chemical or physical property such as a concentration of the component, temperature, pH, ionic strength, or velocity, for example.

A “detector in communication with a sensor chamber” refers to a detector in sufficient optical proximity to the sensor chamber to receive optical signals (e.g., light) from the sensor chamber. A “light source in optical communication” with a chamber refers to a light source in sufficient proximity to the chamber to create a light path from the chamber to a system detector so that optical properties of the chamber or objects contained therein can be detected by the detector.

As used herein, an outlet “intersecting with” a chamber or microchamber refers to an outlet that opens or feeds into a wall or base or top of the chamber or microchamber or into a fluid volume contained by the chamber or microchamber.

As used herein, “superfuse” refers to washing the external surface of an object or sensor (e.g., such as a cell).

As used herein, “cyclic scanning patch-clamp” (CSPC), refers to a method for scanning a patch-clamped cell back and forth through fixed concentration gradients of receptor effectors with control of each cycle in regard of exposure time (tₑₓₚ) clearance time (t_{wash}) and in-between cycle time (tᵣₑₛₜ). This may be applied in other systems, for example to G-protein couple receptors (GPRC) where a preferred method of detection may be fluorescence and alternatively may be electrochemistry, SPR or other methods known in the art for functional receptor protein studies.

The term microfluidic device includes such as microfabricated chips, capillary systems, u-tubes, liquid-filaments and theta-glass on microfluidic flow characterized by low Reynolds number behavior for solution exchange around cells and biosensors.

The term “ligand” as used herein, may refer to a molecule which binds to a receptor which either becomes activated or inactivated. Ligands can act on the receptor as an agonist or antagonist or by modulating the response of the receptor by other agonists or antagonists.

### The System

The invention provides a substrate comprising a chamber for receiving a cell-based biosensor, which comprises a GPCR receptor or ion channel. In one aspect, the system sequentially exposes a cell-based biosensor for short periods of time to one or several ligands which binds to the receptor/ion channel and to buffer without ligand for short periods of time through interdigitated channels of the substrate. For example, selective exposure of a cell biosensor to these different solution conditions for short periods of time can be achieved by exposing the cell-based biosensor to fluid streams that are easily and quickly switched via pressure changes and which alternate delivery of one or several ligands and buffer.

The invention further provides a substrate, which comprises a circular chamber for receiving a sensor, comprising a cylindrical wall and a base. In one aspect, the substrate comprises a plurality of channels comprising opening radially disposed about the circumference of the wall of the chamber (e.g., in a spokes-wheel configuration), for holding a cell, e.g., like a patch-clamp. Preferably, the substrate also comprises at least one output channel for draining waste from the chamber. In one aspect, at least one additional channel delivers buffer to the chamber. Preferably, the angle between the at least one additional channel for delivering buffer and the output channel is greater than 10°. More preferably, the angle is greater than 90°. The channel “spokes” may all lie in the same plane, or at least two of the spokes may lie in different planes.

Rapid, programmed, sequential exchange of solutions in the chamber is used to alter the solution environment around a sensor placed in the chamber. For example, there may be an output channel for each channel for delivering sample/buffer. The number of channels for delivering also can be varied, e.g., to render the substrate suitable for interfacing with an industry standard microtiter plate. For example, there may be 96 to 1024 channels for delivering samples. In another aspect, there may be an additional, equal number of channels for delivering buffer (e.g., to provide interdigitating fluid streams of sample and buffer).

The invention also provides a multi-layered substrate for changing the solution environment around a sensor, comprising: a first substrate comprising channels for delivering fluid to a sensor; a filter layer for retaining one or more sensors which is in proximity to the first substrate; and a second substrate comprising a waste reservoir for receiving fluid from the filter layer. One or more sensors can be provided between the first substrate and the filter layer. In one aspect, at least one of the sensors is a cell. Preferably, the system further comprises a mechanism for creating a pressure differential between the first and second substrate to force fluid flowing from channels in the first substrate through the filter and into the waste reservoir, e.g., providing rapid fluid exchange through the filter (e.g., sensor) layer.

The invention provides systems and methods for generating a high electrical resistance seal between a cell and a surface defining an opening that couples the cell to an electrode compartment or channel. When the cell membrane is sealed against the surface, the cell membrane is in electrical communication with an electrode within the electrode compartment or channel.

In one aspect, the invention provides modified surfaces for optimising the seals between a cell and surface which couples the cell to an electrode compartment or channel. For example, in one aspect, the surface is nonplanar and creates a stress on the cell that creates a tighter seal against the surface. Preferably, the surface is protruded. The surface defining the opening can be part of an on chip patch clamp device, such as an aperture patch clamp array device, or can be the tip of a patch clamp micropipette. The surface may also be an opening in a surface, for example, an opening to a microchannel. The microchannel may be, for example, a microfluidic channel in the substrate. Preferably, the electrical resistance generated when the seal is formed is at least 100 Mohm, at least 1 Gohm, at least 10 Gohm, or at least 100 Gohm.

A sensor may also be trapped at the intersection of trapping channels with a fluidic channel. These trapping sites may be arrayed, and after an experiment, trapped sensors may be expelled by applying positive pressure to the trapping channels so that new or fresh sensors may be trapped for subsequent experiments. All systems described herein may be used one time or multiple times.

The invention also provides an on chip patch clamp device comprising a sensor chamber comprising a non-planar element for maximizing the electrical resistance of a seal formed between a cell and opening of the sensor chamber. In one embodiment, the opening is an opening of a channel wherein for example, the channel is an electrode compartment or channel or contains an electrode. In this aspect, the sensor chamber (sensor channel) defines the electrode compartment or channel, comprising one or more electrical elements at the base of the chamber and an electrolyte solution separating the cell and preventing direct contact between the cell and one or more electrical elements. In one aspect, the non-planar element in the sensor chamber is pyramidal-shaped, conical, elliptical, or toroidal. In another aspect, the nonplanar element comprises a recession for receiving the cell. Preferably, the on chip patch clamp device is an array device comprising a plurality of sensor chambers or channels, and at least one of the sensor chambers comprises a non-planar element. More preferably, substantially all of the sensor chambers comprise non-planar elements.

In one aspect, the sensor channel defines a passageway comprising one or more openings of electrode channels, e.g., the surface of the sensor channel defines the openings of the electrode channels, which couple the sensor to form a high electrical resistance seal at the surface. The electrode channels comprise electrodes.

In another aspect of the invention, the surface defining the opening which couples the cell membrane to the electrode compartment or channel or a cell holding channel or compartment (e.g., cell positioning channel or compartment, or a sensor positioning channel) is modified to provide a surface chemistry that optimises the formation of a high electrical resistance seal at the surface. Preferably, the surface comprises hydrophilic molecules or is treated to be rendered hydrophilic. For example, the surface can be exposed to chemical washing, using an RCA procedure or chemical agents, such as peroxides, ammonia, or nitric acid.

In one preferred embodiment, a surface so treated is the surface of an on chip patch clamp device, such as a patch clamp array device. Preferably, the electrical resistance generated when a seal is formed at such a surface is at least 100 Mohm, at least 1 Gohm, at least 10 Gohm, or at least 100 Gohm.

The invention also provides systems (e.g., microfluidic chips or biosensors) comprising substrates that include one or more sensor chambers or channels for receiving one or more cells. The sensor chambers may form electrode compartment or channels (e.g., as in an on chip patch clamp device) or may receive cells for positioning the cells in proximity to electrode compartment or channels. The substrate may comprise one more microchannels for delivering cells to appropriate sensor chambers. One or more of: pressure, optical tweezers, electroosmosis, dielectrophoresis, and ac or dc currents, may be used to route a cell from a microchannel to an appropriate sensor chamber.

Preferably, the substrate comprises at least one fluid source for providing a fluid stream in proximity to one or more cells in the sensor chamber(s) or channel(s). The fluid stream is used to establish and/or maintain a high electrical resistance seal between a cell and a surface defining an opening for separating the cell from an electrode compartment or channel (e.g., reservoir or channel). In one aspect, the fluid stream is delivered through a microchannel which comprises an outlet which opens into the sensor chamber. In another aspect, the substrate comprises a plurality of microchannels, each having an outlet for delivering fluid streams into a sensor chamber. Preferably, the system comprises a fluid controlling mechanism for controlling hydrostatic pressure at one or more outlets. Hydrostatic pressure at one or more channels can be varied by a processor in communication with the system according to programmed instructions and/or in response to a feedback signal. In one aspect, hydrostatic pressure at each of the plurality of channels is different.

In another aspect, aqueous streams in the same closed channel were collimated laminar streams. The streams may be switched by the application of pressure, negative and/or positive. Thus, for example, a biosensor may be exposed to streams of different content rapidly by switching the streams using differences in the applied pressures.

In one aspect, the longitudinal axes of the electrode channels are substantially parallel. The channels can be arranged in a linear array, in a two-dimensional array, or in a three-dimensional array. Electrode channels, cell holding channels, treatment chambers or channels, sensor chambers or channels, reservoirs, and/or waste channels, and can be interfaced with container(s) or multi-well plate(s) or channels with electrodes. In one aspect, the system comprises at least one input channel for delivering at least one fluid stream into a sensor chamber and at least one output or drain channel for removing fluid from the sensor chamber. In another aspect, output channels can overly input channels (e.g., in a three-dimensional configuration). Preferably, the longitudinal axis of at least one output or drain channel is parallel, but lying in a different plane, relative to the longitudinal axis of at least one input channel. By applying a positive pressure to an input channel at the same time that a negative pressure is applied to an adjacent output or drain channel, a U-shaped fluid stream can be generated within the chamber. The U-shaped fluid streams can be used to create pressure against cells to position and/or seal cells against surfaces which couple the cells to an electrode compartment or channel. Openings to channels may be used as a positioner of a cell or cell-like structure and as a patch device.

In one aspect, one or more fluid streams are used to create a high electrical resistance seal between one or more cells in the sensor chamber (sensor chamber) and one or more surfaces defining openings which separate the cell(s) from electrode compartment or channel(s). For example, fluid streams are used to create high electrical resistance seals between cells and patch clamp micropipettes that are positioned in proximity to the sensor chamber (either by moving the sensor chamber, moving the micropipettes or by moving both the sensor chamber and micropipettes). By controlling the direction of a fluid stream and pressure applied through the fluid stream, a seal with high electrical resistance (e.g., greater than 100 Mohm, and preferably, greater than 1 Gohm) is created.

The invention further provides a method for generating a high electrical resistance seal between a cell membrane and a surface defining an opening for coupling a cell to an electrode compartment or channel or a cell holding compartment or channel. The method comprises exposing the cell to a fluid stream to push the cell against the surface and to obtain a high electrical resistance seal at the surface. Preferably, the seal is maintained for a prolonged period of time, e.g., greater than about 20 minutes, greater than about 30 minutes, greater than about an hour, greater than about 2 hours, or greater than about 5 hours.

The seal may be enhanced by providing a modified surface as described above (e.g., by providing a non-planar or protruded surface, and/or by rendering the surface hydrophilic). Suction or one or more voltages may be applied at the opening to further maximize the electrical resistance of the seal.

In one aspect, the seal created establishes communication (e.g., electrical or other) between a cell membrane and an electrode in the electrode compartment or channel, enabling electrical properties of the cell membrane to be measured. In one aspect, the method is used to obtain patch clamp recordings. Electrical properties recorded may be used to monitor one or more cellular responses and/or cellular properties including, but not limited to: cell surface area, cell membrane stretching, ion-channel permeability, release of internal. vesicles from a cell, retrieval of vesicles from a cell membrane, levels of intracellular calcium, ion-channel induced electrical properties (e.g., current, voltage, membrane capacitance, and the like), or viability.

Any of the systems described above can further comprise a pressure control device for controlling positive and negative pressure applied to at least one microchannel of the substrate. In systems where substrates comprise both delivery channels as well as output channel(s), the system preferably further comprises a mechanism for applying a positive pressure to at least one delivery channel while applying a negative pressure to at least one output channel. Preferably, hydrostatic pressure at least one of the channels can be changed in response to a feedback signal received by the processor.

The system can thus regulate when, and through which channel, a fluid stream is withdrawn from the chamber. For example, after a defined period of time, a fluid stream can be withdrawn from the chamber through the same channel through which it entered the system or through a different channel. When a drain channel is adjacent to a delivery channel, the system can generate a U-shaped fluid stream, which can efficiently recycle compounds delivered through delivery channels.

The system can also regulate fluid streams within a channel. For example, by applying pressure to produce laminar, collimated fluid streams that may be switched by the application of differential pressure as described herein. This may be done in a channel or in an open volume. The system may have a number of suitable pressure sources. For example, the system may have from between 1 and 10 pressure sources or more. In one system, there may be one or more sources providing positive pressure and one or more providing negative pressure. Exemplary pressure source arrangements are described infra in relation to certain embodiments, examples and drawings.

As described above, multiple delivery channel configurations can be provided: straight, angled, branched, fish-bone shaped, and the like. In one aspect, each delivery channel comprises one or more intersecting channels whose longitudinal axes are perpendicular to the longitudinal axis of the delivery channels. In another aspect, each delivery channel comprises one or more intersecting channels whose longitudinal axes are at an angle with respect to the delivery channel.

In general, any of the channel configurations described above are interfaceable with containers for delivering samples to the reservoirs or sample inlets (e.g., through capillaries or tubings connecting the containers with the reservoirs/inlets). In one aspect, at least one channel is branched, comprising multiple inlets. Preferably, the multiple inlets interface with a single container. However, multiple inlets also may interface with several different containers.

Further, any of the substrates described above can be interfaced to a multi-well plate (e.g., a microtiter plate) through one or more external tubings or capillaries. The one or more tubings or capillaries can comprise one or more external valves to control fluid flow through the tubings or capillaries. In one aspect, a plurality of the wells of the multi-well plates comprise known solutions. The system also can be interfaced with a plurality of microtiter plates; e.g., the plates can be stacked, one on top of the other. Preferably, the system further comprises a micropump for pumping fluids from the wells of a microtiter plate or other suitable container(s) into the reservoirs of the substrate. More preferably, the system programmably delivers fluids to selected channels of the substrate through the reservoirs.

In one aspect, a system according to the invention further comprises a detector in communication with a sensor chamber or channel for detecting sensor responses. For example, the detector can be used to detect a change in one or more of: an electrical, optical, or chemical property of the sensor. In one aspect, in response to a signal from the detector, the processor alters one or more of: the rate of exposing, number of exposures and pressure on one or more channels.

In one aspect, the system provides a substrate comprising a plurality of microchannels fabricated thereon. There are one or more sensor channels, wherein a plurality of electrode channels outlets intersect with, or feed into, a sensor channel, which may comprise one or more sensors. In a preferred aspect, the sensor chamber comprises a cell-based biosensor in electrical communication with an electrode and the detector detects changes in electrical properties of the cell-based biosensor.

The system preferably also comprises a processor for implementing system operations including; but not limited to: controlling fluid flow through one or more channels of the substrate, controlling the operation of valves and switches that are present for directing fluid flow, recording sensor responses detected by the detector, and evaluating and displaying data relating to sensor responses. Preferably, the system also comprises a user device in communication with the system processor which comprises a graphical interface for displaying operations of the system and for altering system parameters. The system is further described below.

### The Substrate

In a preferred aspect, the system comprises a substrate that delivers solutions to a sensor chamber or channel wherein the sensor channel is adapted to receive one or more sensors. The substrate can be configured as a two-dimensional (2D) or three-dimensional (3D) structure, as described further below. The substrate, whether 2D or 3D, generally comprises a plurality electrode microchannels whose outlets intersect with a sensor chamber and whose openings are adapted to receive one or more sensors. The base of the sensor chamber can be optically transmissive to enable collection of optical data from the one or more sensors placed in the sensor chamber.

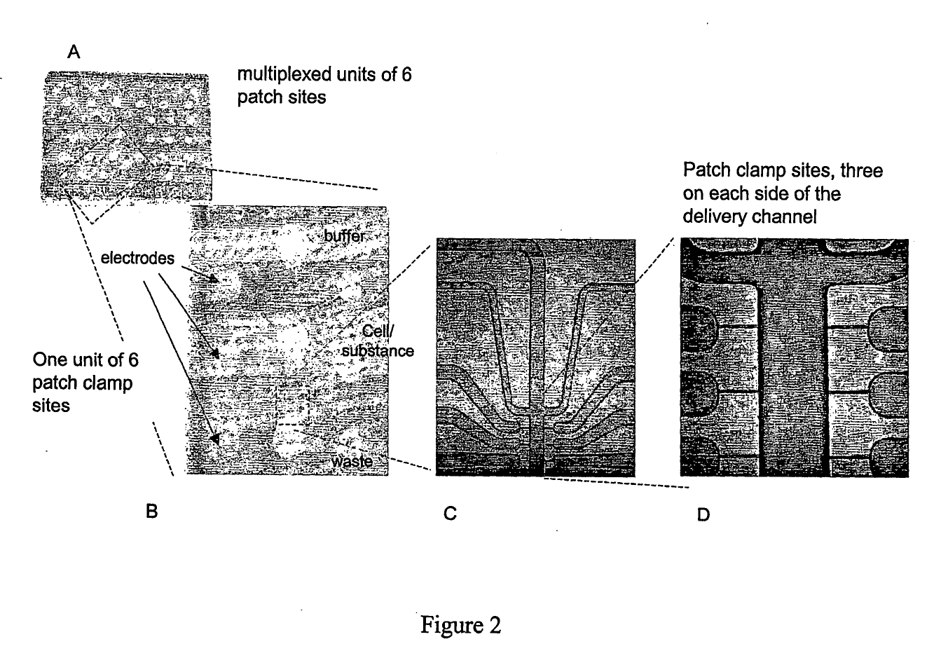

Each microchannel comprises at least one inlet (e.g., for receiving a sample or a buffer). Preferably, the inlets receive solution from reservoirs (e.g., shown as circles in FIGS. 2A and B) that conform in geometry and placement on the substrate to the geometry and placement of wells in an industry-standard microliter plate. The substrate is a removable component of the system and therefore, in one aspect, the invention provides kits comprising one or more substrates for use in the system, providing a user with the option of choosing among different channel geometries.

Non-limiting examples of different substrate materials include crystalline semiconductor materials (e.g., silicon, silicon nitride, Ge, GaAs), metals (e.g., Al, Ni), glass, quartz, crystalline insulators, ceramics, plastics or elastomeric materials (e.g., silicone, EPDM and Hostaflon), other polymers (e.g., a fluoropolymer, such as Teflon®, polymethylmethacrylate, polydimethylsiloxane, polyethylene, polypropylene, polybutylene, polymethylpentene, polystyrene, polyurethane, polyvinyl chloride, polyarylate, polyarylsulfone, polycaprolactone, polyestercarbonate, polyimide, polyketone, polyphenylsulfone, polyphthalamide, polysulfone, polyamide, polyester, epoxy polymers, thermoplastics, and the like), other organic and inorganic materials, and combinations thereof.

Microchannels can be fabricated on these substrates using methods routine in the art, such as deep reactive ion etching (described further below in Example 1). Channel width can vary depending upon the application, as described further below, and generally ranges from about 0.1 μm to about 10 mm, preferably, from about 1 μm to about 150 μm, while the dimensions of the sensor chamber generally will vary depending on the arrangement of electrode channel outlets feeding into the chamber. For example, where the outlets are substantially parallel to one another (e.g., as in FIGS. 2B-D). In one aspect, where a whole cell biosensor is used as a sensor in the sensor chamber, the width of one or more outlets of the microchannels is at least about the diameter of the cell. Preferably, the width of each of the outlets is at least about the diameter of the cell. Preferably, the base of the sensor chamber also is optically transmissive, to facilitate the collection of optical data from the sensor.

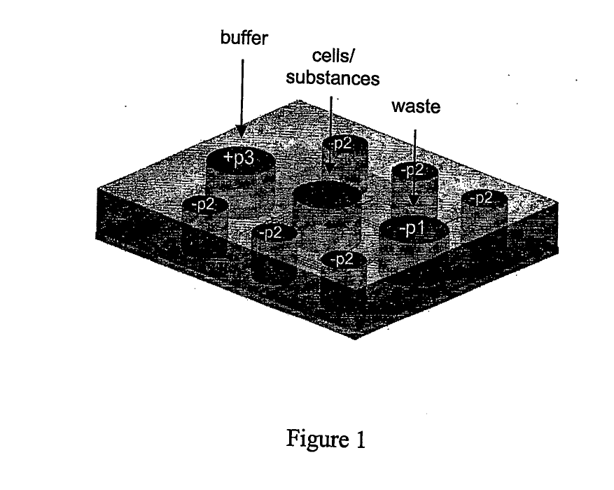

FIG. 1 is a 3-D perspective illustration of the reservoirs comprising a 6-patch site microfluidic patch clamp chip. The reservoir 200 is adapted or configured to hold a buffer solution and to transmit the positive pressure denoted as +p3 for moving the buffer solution through the delivery channel. The center reservoir 210 is adapted for loading sensors or substances to be flowed through the delivery channel. The reservoir 220 is adapted for the waste and transmits the negative pressure denoted as −p1 for controlling the delivery of sensors and substances or ligands to the patch sites. It may also be the site for placement of the common ground electrode for detecting transmembrane current responses at the patch sites. The reservoirs 230A-F is adapted to receive or comprise an electrode and to transmit. negative pressure denoted as −p2 for controlling cell immobilization at the individual patch sites.

FIG. 2A depicts a photograph of a microfluidic patch-clamp chip incorporating 4 multiplexed 6-patch site units. There are four buffer reservoirs 200A-D, four cell/substance reservoirs 210A-D, and four waste reservoirs 220A-D. The reservoirs communicating with the 24 patch sites on the chip are located in the 1st, 3rd, 4th and 6th rows of the chip, with a representative patch clamp reservoir 230 labeled in the first row and last column of the chip.

FIG. 2B depicts a single 6-patch site unit isolated and magnified from the 4-unit multiplex chip of FIG. 2A. It represents the 6-patch site unit on the lower left corner of the multiplex chip of FIG. 2A, which has been magnified and rotated counterclockwise 90 degrees. The buffer reservoir 200A has an outlet channel that bifurcates around the cell/substance reservoir 210A, subchannel 120A fluidly connected with the left side of the delivery channel 100, and subchannel 120B fluidly connected with the right side of the delivery channel 100. The cell/substance (inlet) reservoir 210A has an inlet channel 110 that courses in a set of four reversing right angles toward the buffer reservoir B, then turns toward and is fluidly connected to the delivery channel 100. The patch site reservoirs 230A-F are in fluid communication with individual channels 130A-F that are fluidly connected with the delivery channel 100. The waste reservoir 220A is fluidly connected to the delivery channel 100.

FIG. 2C is a schematic showing the arrangement of the inflow channels and the patch channels fluidly connected with the delivery channel 100, isolated and magnified from the corresponding area outlined in FIG. 2B. The inlet channel 110 is adapted to transmit sensors or substances into the delivery channel 100 from the reservoir 210A. The two flanking channels 120A and 120B are the bifurcated inlet channels in fluid communication from the buffer reservoir 200A. The six downstream channels 130A-F (three on each side of the delivery channel 100) are in fluid communication with the individual reservoirs 230A-F that hold the detection electrodes and provide the negative pressure −p2 for immobilizing the cells at the patch sites 150.

FIG. 2D is a schematic further magnifying and isolating the arrangement of the six patch sites 150 in relation to the delivery channel 100. Each patch channel 130A-F further narrows into a channel oriented at a right angle to the delivery channel 100, and terminates into a patch site 150.

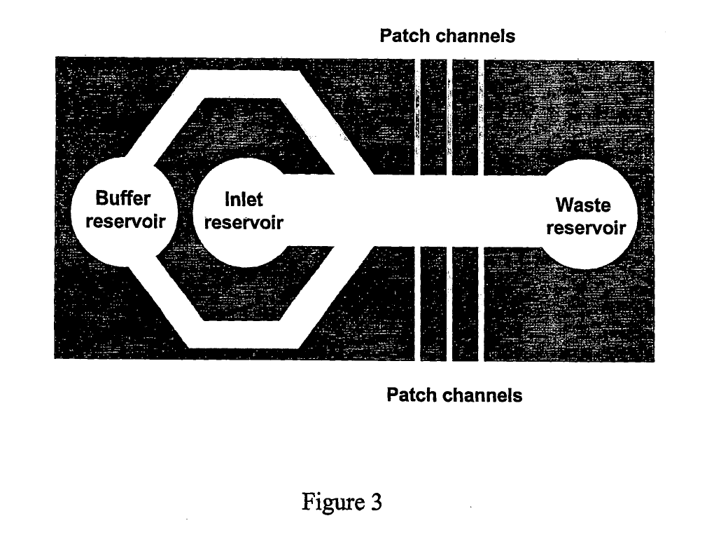

In reference to FIG. 3, a schematic drawing is shown. The functional components of the microfluidic six-patch site unit are depicted, wherein a buffer reservoir 200 is fluidly connected with the delivery channel 100 via the bifurcated channels 120A and 120B. Channel 120A enters on one side of the delivery channel 100, and channel 120B enters on the other side of the delivery channel 100. There are 3 patch channels 130 terminating into patch sites 150, on each side of the delivery channel 100. The delivery channel 100 empties into the waste reservoir 220.

### The Sensor

Cell-Based Biosensors

The system can be used in conjunction with a cell-based biosensor to monitor a variety of cellular responses. The biosensor can comprise a whole cell or a portion thereof (e.g., a cell membrane patch) which is positioned in the sensor chamber using a mechanism for holding a sensor (which may be stationary or movable) such as a pipette, capillary, or column connected to a positioner, such as a micropositioner, a nanopositioner or a micromanipulator, or an optical tweezer, or by controlling flow or surface tension, thereby exposing the cell-based biosensor to solution in the chamber. The biosensor can be scanned across the various channels of the substrate by moving the substrate, e.g., changing the position of the channels relative to the biosensor, or by moving the cell (e.g., by scanning the micropositioner or by changing flow and/or surface tension). The biosensor may also be exposed to, for example, candidate drugs and/or other compounds and compositions.

In one aspect, the cell-based biosensor comprises an ion channel and the system is used to monitor ion channel activity. Suitable ion channels include ion channels. gated by voltage, ligands, internal calcium, other proteins, membrane stretching (e.g., lateral membrane tension) and phosphorylation (see e.g., as described in Hille B., In Ion Channels of Excitable Membranes 1992, Sinauer, Sunderland, Mass., USA). In another aspect, the ion-gated channel is a voltage-gated channel. Voltage-gated channels open in response to a threshold transmembrane voltage. Voltage-gated sodium, potassium, and calcium channels are all essential for conducting an action potential (or a nerve pulse) down an axon and to another nerve cell (or neuron). These ion channels typically comprise a transmembrane sequence with a lysine and/or arginine-rich S4 consensus sequence. The positive amino acids within the S4 sequence are thought to “sense” voltage across a cell membrane, causing an ion channel containing the sequence to either open or close under different voltage conditions.

In another aspect, the ion channel in the cell-based biosensor is a ligand-gated channel. Ligand-gated channels gate (open or close) in response to ligand binding. There are two types of ligand-gated channels, those gated when bound by ligands inside the cell and those gated by ligands outside the cell. Ion channels gated by ligands from outside of the cell are very important in chemical synaptic transmission. These types of ion channels are gated by neurotransmitters, which are the small molecules that actually carry the signal between two nerve cells. Ion channels gated from the inside of the cell are generally controlled by second messengers, which are small signaling molecules inside the cell. Intracellular calcium ions, cAMP and cGMP are examples of second messengers. The most common calcium-gated channel is the calcium-gated potassium channel. This ion channel can generate oscillatory behavior (e.g., for frequency tuning of hair cells in the ear) upon changes in membrane voltage when placed in a positive feedback environment.

In yet another aspect, the ion channel is gated by another protein. Certain signaling proteins have been found to directly gate ion channels. One example of this is a potassium channel gated by the beta-gamma subunit of the G protein, which is a common signaling protein activated by certain membrane receptors.

In a further aspect, the ion channel is gated by phosphorylation. Phosphorylation can be mediated by a protein kinase (e.g., a serine, threonine, or tyrosine kinase), e.g., as part of a signal transduction cascade.

In still a further aspect, the cell-based biosensor comprises a mechanotransduction channel that can be directly gated by a mechanical trigger. For example, the cell-based biosensor can comprise the cation channel of an inner ear hair cell, which is directly gated by a mechanical vibration such as sound. Bending of the hair bundle in a particular direction will affect the probability of channel gating, and therefore, the amplitude of a depolarizing receptor current.

In another aspect, the cell-based biosensor comprises a receptor, preferably, a receptor involved in a signal transduction pathway. For example, the cell-based biosensor can comprise a G Protein Coupled Receptor or GPCR, glutamate receptor, a metabotropic receptor, a hematopoietic receptor, or a tyrosine kinase receptor. Biosensors expressing recombinant receptors also can be designed to be sensitive to drugs which may inhibit or modulate the development of a disease.

Suitable cells which comprise biosensors include, but are not limited to: neurons; lymphocytes; macrophages; microglia; cardiac cells; liver cells; smooth muscle cells; and skeletal muscle cells. In one aspect, mammalian cells are used; these can include cultured cells such as Chinese Hamster Ovary Cells (CHO) cells, NIH-3T3, and HEK-293 cells and can express recombinant molecules (e.g., recombinant receptors and/or ion channels). However, bacterial cells (E. coli, Bacillus sp., Staphylococcus aureus, and the like), protist cells, yeast cells, plant cells, insect and other invertebrate cells, avian cells, amphibian cells, and oocytes, also can be used, as these are well suited to the expression of recombinant molecules. Cells generally are prepared using cell culture techniques as are know in the art, from cell culture lines, or from dissected tissues after one or more rounds of purification (e.g., by flow cytometry, panning, magnetic sorting, and the like).

Non-Cellular Sensors

In one aspect, the sensor comprises a sensing element, preferably, a molecule which is cellular target (e.g., an intracellular receptor, enzyme, signaling protein, an extra cellular protein, a membrane protein, a nucleic acid, a lipid molecule, etc.), which is immobilized on a substrate. The substrate can be the base of the sensor chamber itself; or can be a substrate placed on the base of the chamber, or can be a substrate which is stably positioned in the chamber (e.g., via a micropositioner) and which is moveable or stationary.

The sensor may consist of one or several layers that can include any combination of a solid substrate; one or more attachment layers that bind to the substrate, and a sensing molecule for sensing compounds introduced into the sensor chamber from one or more channel outlets. Suitable sensors according to the invention, include, but are not limited to, immunosensors, affinity sensors and ligand binding sensors, each of which can respond to the presence of binding partners by generating a measurable response, such as a specific mass change, an electrochemical reaction, or the generation of an optical signal (e.g., fluorescence, or a change in the optical spectrum of the sensing molecule). Such sensors are described in U.S. Pat. No. 6,331,244, for example, the entirety of which is incorporated by reference herein.

In one aspect, the sensor comprises a microelectrode which is modified with a molecule which transports electrons. In response to a chemical reaction caused by contact with one or more compounds in an aqueous stream from one of the microchannels, the molecule will produce a change in an electrical property at the electrode surface. For example, the molecule can comprise an electron-transporting enzyme or a molecule which transduces signals by reduction or oxidation of molecules with which it interacts (see, e.g., as described in, Gregg, et al., J. Phys. Chem. 95: 5970-5975, 1991; Heller, Acc. Chem. Res. 23(5): 128-134, 1990; In Diagnostic Biosensor Polymers. ACS Symposium Series. 556; Usmani, A M, Alcmal, N; eds. American Chemical Society; Washington, D.C.; pp. 47-70, 1994; U.S. Pat. No. 5,262,035). Enzymatic reactions also can be performed using field-effect-transistors (FETs) or ion-sensitive field effect transistors (ISFETs).

In another aspect, the sensor comprises a sensing molecule immobilized on a solid substrate such as a quartz chip in communication with an electronic component. The electronic component can be selected to measure changes in any of: voltage, current, light, sound, temperature, or mass, as a measure of interaction between the sensing element and one or more compounds delivered to the sensor chamber (see, as described in, Hall, Int. J. Biochem. 20(4): 357-62, 1988; U.S. Pat. No. 4,721,677; U.S. Pat. No. 4,680,268; U.S. Pat. No. 4,614,714; U.S. Pat. No. 6,879,11). For example, in one aspect, the sensor comprises an acoustic wave biosensor or a quartz crystal microbalance, on which a sensor element is bound. In this embodiment, the system detects changes in the resonant properties of the sensor upon binding of compounds in aqueous streams delivered from the microchannels to the sensor element.

In another aspect, the sensor comprises an optical biosensor. Optical biosensors can rely on detection principles such as surface plasmon resonance, total internal reflection fluorescence (TIRF), critical angle refractometry, Brewster Angle microscopy, optical waveguide lightmode spectroscopy (OWLS), surface charge measurements, and evanescent wave ellipsometry, and are known in the art (see, for example, U.S. Pat. No. 5,313,264; EP-A1-0 067 921; EP-A1-0 278 577; Kronick, et al., 1975, J. Immunol. Meth. 8: 235-240).

For example, for a sensor employing evanescent wave ellipsometry, the optical response related to the binding of a compound to a sensing molecule is measured as a change in the state of polarization of elliptically polarized light upon reflection. The state of polarization is related to the refractive index, thickness, and surface concentration of a bound sample at the sensing surface (e.g., the substrate comprising the sensing element). In TIRF, the intensity and wavelength of radiation emitted from either natively fluorescent or fluorescence-labelled sample molecules at a sensor is measured. Evanescent wave excitation scattered light techniques rely on measuring the intensity of radiation scattered at a sensor surface due to the interaction of light with sensing molecules (with or without bound compounds). Surface plasmon resonance (SPR) measures changes in the refractive index of a layer of sensor molecules close to a thin metal film substrate (see, e.g., Liedberg, et al., 1983, Sensors and Actuators 4: 299; GB 2 197 068). Each of these sensing schemes can be used to provide useful sensors according to the invention.

In yet another aspect, the sensor comprises a sensing molecule associated with a fluorescent semiconductor nanocrystal or a Quantum Dot™ particle. The Quantum Dot particle has a characteristic spectral emission which relates to its composition and particle size. Binding of a compound to the sensing element can be detected by monitoring the emission of the Quantum Dot particle (e.g., spectroscopically) (see, e.g., U.S. Pat. No. 6,306,614.

The sensor further can comprise a polymer-based biosensor whose physical properties change when a compound binds to a sensing element on the polymer. For example, binding can be manifested as a change in volume (such as swelling or shrinkage), a change in electric properties (such as a change in voltage or current or resonance) or in optical properties (such as modulation of transmission efficiency or a change in fluorescence intensity).

It should be obvious to those of skill in the art that a variety of different types of sensors may be adapted for use in present invention, and the examples above are intended to be non-limiting.

In general, the measurement outputs of one or more sensors are connected to a control and evaluating device which is in electrical communication with a detection device and/or system processor. The control and evaluating device can be integrated with the substrate of the sensor and/or with the base of the sensing chamber. The control and evaluating device can comprise various electronic components such as microprocessors, multiplexers, 10 units, etc. (see, e.g., as described in U.S. Pat. No. 6,280,586).

In a preferred aspect, the substrates according to the invention are adapted for microfluidic transport of sample and/or buffer to a sensor chamber.

Samples (e.g., drugs, etc.) contained in sample-well plates (e.g., industry-standard microtiter plates such as 96-well plates) are manipulated and transferred, preferably, using robotic automated array pipettors as are known in the art (see, e.g., Beckman's Biomek 1000 & 2000 automated workstations, available from Beckman Coulter, Inc., Fullerton, Calif.).

The system can be programmed to deliver cells from the cell treatment chamber at selected time periods based on control experiments monitoring uptake of chemicals and molecules by cells. Alternatively, the system can monitor the phenotype of cells and deliver cells when a certain phenotype is expressed. For example, in one aspect, the cell treatment chamber is in communication with an optical sensor which provides information relating to optical properties of the cell to the system processor, and in response to optical parameters indicating expression of a particular phenotype, the system can trigger release of the cell from the cell treatment chamber. Optical parameters can include the uptake of a fluorescent reporter molecule or optical parameters identified in control experiments.

The combination of on-chip electroporation with microfluidics and patch clamp (or other methods for monitoring cell responses) facilitates screening for molecules (e.g., ligands or drugs) which modulate the activity of intracellular targets. In one aspect, the system is used to deliver a cell-impermeant molecule into the interior of a cell by transiently electroporating the cell. In this way, the molecule can be introduced to intracellular receptors, intracellular proteins, transcriptional regulators, and other intracellular targets. The cell can be delivered to the sensor chamber and the response of the cell can be monitored (e.g., by patch clamp or by fluorescence, if the molecule is tagged with a fluorescent label). Alternatively, the sensor chamber can be modified to perform both treatment and response detection.

In a further aspect, the system can be modified to perform electroporation by scanning. For example, a cell can be repeatedly electroporated as it is being translated or scanned across a plurality of different fluid streams containing different compounds. In one aspect, pores are introduced into one or more cells as they come into contact with a sample stream, enabling compounds in the sample stream to be taken up by the cell.

High Electrical Resistance Seals in a Biosensor

The invention further provides a system for maximizing the electrical resistance of a seal between a cell membrane, and the opening of a surface separating the cell membrane from an electrode compartment or channel, maximizing the electrical resistance of a seal between the cell membrane and the opening. The invention also provides a method for providing an optimal configuration at the opening by providing one or more of: an optimal geometry and/or surface topography at the surface defining the opening; optimal surface chemistry at the surface defining the opening (e.g., providing hydrophilic groups at the surface); and fluid flow in proximity to a cell membrane positioned in proximity to the opening.

The systems and methods of the present invention may be used for techniques such as internal perfusion of oocytes, patch clamp electrophysiology, brain slice recording, receptor-ligand interactions on cell surfaces, calcium imaging studies, confocal microscopy, and in vivo microdialysis, for example. The system of the present invention may also be used to measure properties of ligand-gated ion channels, voltage-gated ion channels, G-protein coupled receptors, activities across a synapse, molecular transporters, cell-to-cell interactions and ion pumps, and to screen for modulators (agonists or antagonists) of these biomolecules.

For geometrical properties of a surface separating a cell from an electrode compartment or channel see for example U.S. application Ser. No. 10/345,107 and U.S. application Ser. No. 10/645,834, which are hereby incorporated by reference in their entirety.

In one aspect, therefore, the invention provides a method for maximizing seal resistance between a cell and such an opening, thereby to maximize the efficiency of patch clamp recordings. Empirically, it was found that the attractive interaction between a lipid membrane and a surface defining such an opening is maximized when the surface is made hydrophilic. The more hydrophilic the surface, the stronger is the attractive interaction. A strong attraction provides a larger contact area and a smaller separation distance between the two surfaces and results in higher seal resistance.

A strong attraction provides a larger contact area the surface interaction energies between the tip and a cell being analyzed is sufficient to deform the cell.

### Systems, System Components, and Methods for Increasing the Efficiency of a Patch Clamp Recording Device

Provided herein are methods for modulating, controlling, preparing, or studying receptors, comprising providing a substrate, the substrate comprising a chamber comprising a cell-based biosensor comprising a receptor which is activated by an agonist; and a plurality of delivery channels delivering agonist, antagonist, or both agonist and antagonist; and sequentially exposing the biosensor to two or more different fluid streams.

According to one aspect, the chamber comprises at least one of a buffer, a sample, an agonist, anantagonist, or a combination thereof. In one aspect, the exposing is selectively exposing the biosensor to a selected concentration of a sample. In a related aspect, the exposing is selectively for a selected time. In another aspect, the system further comprises providing to the channels one or more buffers.

In yet another aspect, the system further comprises exposing the biosensor to the one or more buffers. According to a related aspect, the exposing the biosensor to one or more buffers is interspersed between the exposing to one or more samples. In another related aspect, the exposing to one or more buffers is a wash period. In yet another related aspect, the exposing to one or more buffers is a rest period. In still another aspect, the system further comprises the exposing to one or more buffers is a wash and a rest period.

In one aspect, a rest period in buffer is between a series of sample exposures and interdigitated by one or more wash periods in buffer.

In another aspect, selectively exposing the biosensor to streams of buffer and sample. According to a related aspect, selectively exposing the biosensor to alternating streams of buffer and sample. In another related aspect, the receptors are exposed to ligand solutions in order of increasing concentrations. In another related aspect, the receptors are exposed to ligand solutions in order of decreasing concentrations. In a related aspect, the receptors are exposed to ligand solutions in order of increasing concentrations followed by exposure to ligand solutions in order of decreasing concentrations. In yet another related aspect, the receptors are exposed to ligand solutions in order of decreasing concentrations followed by exposure to ligand solutions in order of increasing concentrations. In yet another related aspect, the receptors are exposed to washing solution after ascending or descending exposures of modulator.

In another aspect, the agent is selected from a candidate drug; a known drug; a suspected carcinogen; a known carcinogen; a candidate toxic agent, a known toxic agent; and an agent that acts directly or indirectly on ion channels.

According to one aspect, the method for studying is a method for studying the memory properties of a receptor. According to another aspect, the memory functions are short-term, medium-term, or long-term memory functions. In a related aspect, the effects of a drug on memory properties of a biosensor are studied.

In another aspect, the exposing step is performed by moving the substrate or a sensor or both the substrate and the sensor relative to at least one channel outlet. In another related aspect, the exposing further comprises producing pressure drops across one or more channels.

According to one aspect, the same sample is provided to a plurality of channels. In a related aspect, different concentrations of the sample are provided to the plurality of channels.

In another aspect, the system further comprises generating a dose-response curve for the sample.

In another aspect, the cell-based biosensor comprises an ion-channel. In a related aspect, the receptor comprises a G-protein coupled receptor. In another related aspect, the cell-based biosensor comprises a recombinantly expressed receptor. In still another related aspect, the recombinantly expressed receptor is an orphan receptor.

In one aspect, the response to the sample is determined by measuring cell surface area. In a related aspect, the response is determined by measuring an electrical property of the cell-based biosensor. In another related aspect, the response is determined by measuring ion-channel permeability properties.

In another aspect, the sample is a modulator of neurotransmitter release.

According to another embodiment, a method of preparing a receptor in a discrete kinetic state is presented. The method comprises sequentially exposing a cell-based biosensor to two or more concentrations of modulator, and alternating resting and washing periods between exposures to modulator, wherein the sequential exposure arrests the biosensor in a pre-determined kinetic state.

According to one aspect, the sequentially exposing ranges from between about 1 ms to about 180 minutes, or from between 1 ins to about 60 minutes, or from between about 1 ms to tens of minutes, or from 1 ms to the death of the cell.

In a related aspect, the resting ranges from between about 1 ins to about 180 minutes, or from between 1 ms to about 60 minutes, or from between about 1 ms to tens of minutes, or from 1 ins to the death of the cell.

In another related aspect, the washing periods range from between about 1 ms to about 180 minutes, or from between 1 ms to about 60 minutes, or from between about 1 ins to tens of minutes, or from 1 ms to the death of the cell.

In another aspect, the system further comprises determining the molecular memory of a biosensor. In a related aspect, the molecular memory is determined by measuring a dose response of the modulator.

In another aspect, increasing concentrations of modulator are exposed to the biosensor. In related aspect, decreasing concentrations of modulator are exposed to the biosensor.

In one aspect, wherein the modulator is selected from a candidate drug; a known drug; a suspected carcinogen; a known carcinogen; a candidate toxic agent, a known toxic agent; and an agent that acts directly or indirectly on ion channels.

The invention also provides a method for changing an aqueous or other liquid solution environment locally around a nanoscopic or microscopic object (e.g., such as a sensor). The method comprises providing a substrate as described herein and an aqueous or other liquid fluid.

Preferably, fluid streams exiting or merging from the at least two channels are collimated and laminar within the sensor chamber. However, the system can comprise sets of channels (at least two adjacent channels) wherein at least one set delivers collimated laminar streams, while at least one other set delivers non-collimated, laminar streams. In one aspect, the streams flow at different velocities. Fluid can be delivered from the channels to the sensor channel or chamber by a number of different methods, including by electrophoresis and/or by electroosmosis and/or by pumping (e.g., pressure). The laminar streams may be switched to selectively expose the sensors to different fluid streams. The switching may be done, for example, by pressure applied to the streams. For example, switching the pressure drop applied to one stream with respect to the second stream—which may be achieved, for example, either by applying a greater positive pressure to the inlet reservoir of the first stream or by applying a greater negative pressure to the outlet reservoir of the first stream—will cause a fluid displacement of the second stream by the first stream. As a result, a sensor placed originally in the second stream and thus exposed to the solution in the second stream, will become exposed to the solution in the first stream because of the fluid displacement.

In one aspect, the channels can be arranged in a linear array, in a two-dimensional array, or in a three-dimensional array, can comprise, sensor chambers or channels, electrode channels, reservoirs, and/or waste channels, and can be interfaced with container(s) or multi-well plate(s) as described above. In one aspect, output channels can overly input channels (e.g., in a three-dimensional configuration). By applying a positive pressure to an input channel at the same time that a negative pressure is applied to an adjacent output or drain channel, a U-shaped fluid stream can be generated within the chamber. In this way, an object within the chamber can be exposed to a compound in a fluid stream from an inlet channel which can, for example, be recycled by being withdrawn from the chamber through the adjacent output or drain channel. The U-shaped fluid streams can thus be used to create local well-defined regions of fluid streams with specific composition in a large-volume reservoir.

In another aspect, a main fluid channel is perpendicular to one or more electrode channels. Differential pressure may be applied to the channels to aid in the attachment of a biosensor to the channels. Differential pressure may also be used to selectively expose the biosensors to different fluids, for example, buffer and wash solutions or solutions containing one or more solutes, e.g., drugs, drug candidate composition, and the like.

The sensor chamber can comprise a plurality of objects; preferably, each object is selectively exposed to at least two streams. Exposing can be performed by an exposing mechanism controlled by a processor as described above. The sensor chamber can additionally have inlets and outlets for adding and withdrawal of solution. For example, fresh buffer solution can be added by using a peristaltic pump.

In one aspect, the method further comprises modifying one or more exposing parameters, such as the rate of exposing, the direction of exposing, acceleration of exposing, number of exposures, and pressure across one or more channels. Exposing parameters can be modified in response to a feedback signal, such as a signal relating to the response of an object to one or more of aqueous streams.

Hydrostatic pressure at one or more channels also can be varied by the processor according to programmed instructions and/or in response to a feedback signal. In one aspect, hydrostatic pressure at each of the plurality of channels is different.

In another aspect, the viscosity of fluids in at least two of the channels is different. In yet another aspect, fluid within at least two of the channels are at a different temperature. In a further aspect, the osmolarity of fluid within at least two of the channels is different. In a still further aspect, the ionic strength of fluid within at least two of the channels is different. Fluid in at least one of the channels also can comprise an organic solvent. By changing these parameters at different outlets, sensor responses can be optimized to maximize sensitivity of detection and minimize background. In some aspects, parameters also can be varied to optimize certain cell treatments being provided (e.g., such as electroporation or electrofusion).

The invention also provides a method for rapidly changing the solution environment around a nanoscopic or microscopic object, which comprises rapidly exchanging fluid in a sensor chamber or channel comprising the nanoscopic or microscopic object. In one aspect, fluid exchange in the chamber occurs within less than about 1 minute, preferably, with less than about 30 seconds, less than about 20 seconds, less than about 10 seconds, less than about 5 seconds, or less than about 1 second. In another aspect, fluid exchange occurs within milliseconds. In another aspect fluid exchange occurs within nanoseconds. The fluid may be exchanged using selective pressure and switching which fluid stream is in contact with the object.

The method may be used to measure responses of a cell or portion thereof to a condition in an aqueous environment, by providing a cell or portion thereof in the chamber of any of the substrates described above, exposing the cell or portion thereof to one or more aqueous streams for creating the condition, and detecting and/or measuring the response of the cell or portion thereof to the condition. For example, the condition may be a chemical or a compound to which the cell or portion thereof is exposed and/or can be the osmolarity and/or ionic strength and/or temperature and/or viscosity of a solution in which the cell or portion thereof is bathed.

The composition of the bulk solution in the sensor chamber or channel in any of the substrates described above can be controlled, e.g., to vary the ionic composition of the sensor chamber or to provide chemicals or compounds to the solution. For example, by providing a laminar switching system (exposing mechanism), a chemical or a compound, such as a drug, can be added to the sensor chamber during the course of an experiment.

In one aspect, exposure of the cell or portion thereof to the condition occurs in the sensor chamber. However, alternatively, or additionally, exposure of the cell or portion thereof to the condition can occur in a microchamber or in a channel which connects to the sensor chamber via one or more channels or is the connection. The cell or portion thereof can be transferred to the sensor chamber in order to measure a response induced by changing the conditions around the cell.

In one aspect, the invention also provides a method for generating an activated receptor or ion channel in order to detect or screen for antagonists. The method comprises delivering a constant stream of an agonist to a cell-based biosensor in a sensor chamber. Preferably, the cell-based biosensor expresses receptor/ion channel complexes which do not desensitize or which desensitize very slowly. Exposure of the biosensor to the agonist produces a measurable response, such that the receptor is activated each time it passes a microchannel delivering agonist. Preferably, a plurality of the agonist delivering microchannels also comprise antagonist whose presence can be correlated with a decrease in the measurable response (e.g., antagonism) when the cell-based biosensor passes by these microchannels. In one aspect, a plurality of microchannels comprises equal amounts of agonist but different concentrations of antagonist or the laminar streams in one channel comprise equal amounts of agonist but different concentrations of antagonist. Inhibition of the measurable response can thus be correlated with the presence of a particular dose of antagonist. In another aspect, a plurality of microchannels comprise equal amounts of agonist, but one or more, and preferably all of the plurality of microchannels, comprises different kinds of antagonists. In this way the activity of particular types of antagonists (or compounds suspected of being antagonists) can be monitored.

In one aspect, a periodically re-sensitized receptor is provided by switching the laminar streams via pressure to deliver pulses of buffer to the cell-based biosensor, to thereby remove any bound agonist or modulator desensitizing the receptor, before the receptor is exposed to the next channel outlet containing agonists or receptor modulators. In detection of antagonists, the switching of the laminar stream system can also periodically remove the constantly applied agonist. A transient peak response (which is desensitized to a steady state response) is generated when the re-sensitized biosensor is exposed to the agonist. The generation of this peak response can provide a better signal-to-noise ratio in detection of antagonists.

In another aspect, ion-channels in a cell-based biosensor are continuously activated or periodically activated by changing the potential across the cell-membrane. This provides a sensor for detection of compounds or drugs modulating voltage-dependent ion-channels.

Responses measured by the systems or methods will vary with the type of sensor used. When a cell-based biosensor is used, the agonist-, antagonist-, or modulator-induced changes of the following parameters or cell properties can be measured: cell surface area, cell membrane stretching, ion-channel permeability, release of internal vesicles from a cell, retrieval of vesicles from a cell membrane, levels of intracellular calcium, ion-channel induced electrical properties (e.g., current, voltage, membrane capacitance, and the like), optical properties, or viability.

In one aspect, the sensor comprises at least one patch-clamped cell. Thus, a cell or cell membrane fraction are positioned appropriately by the electrode channels and the laminar switching system rapidly and selectively exposes the sensors to the correct fluid streams in parallel if there is more than one electrode channel on one side of a sensor chamber.

The systems and methods according to the invention can be used to perform high throughput screening for ion channel and GPCRs ligands and for drugs or ligands which act directly or indirectly on ion channels or GPCRs. However, more generally, the systems and methods can be used to screen for compounds/conditions, which affect any extracellular, intracellular, or membrane-bound target(s). Thus, the systems and methods can be used to characterize, for example, the effects of drugs on cell. Examples of data that can be obtained for such purposes according to the present invention includes but is not limited to: dose response curves, IC₅₀ and EC₅₀ values, voltage-current curves, on/off rates, kinetic information, thermodynamic information, etc.

In one aspect, the invention provides systems, system components, and methods for performing measurements of the electrical properties of a cell membrane for prolonged periods of time, e.g., greater than about 20 minutes, preferably greater than about one hour, greater than about 2 hours, greater than about 3 hours, greater than about 4 hours, or greater than about 5 hours.

In one aspect, a system according to the invention comprises an electrode compartment or channel (e.g., compartment, reservoir or channel) comprising one or more electrodes, a lumen for receiving an electrolyte solution and for electrically coupling the electrode(s) to a cell membrane, and a surface defining an opening that is in fluid communication with the lumen. In one aspect, the lumen is a channel in a microfluidic device. The channel may be in fluid communication with a sensor chamber. In another aspect, the lumen is part of a sensor chamber for receiving a cell membrane in an on-chip patch clamp device, such as a patch clamp array device. Preferably, the cell membrane is in electrical communication with the electrodes through contact with the electrolyte solution.

As used herein, the electrode(s) and lumen comprising electrolyte solution define an “electrode compartment or channel.” In some instances the electrical elements can form part of the electrode compartment or channel. The surface defining the opening in communication with the lumen serves as a partition between the electrode compartment or channel and cell, and more particularly, between the electrode compartment or channel, and a bath solution in which the cell membrane resides.

Suitable surfaces include glass (e.g., when the surface is part of a patch clamp micropipette) or a polymer such as a carbon-based polymer, a silicone-based polymer, a plastic, and modified or treated forms thereof.

The surface defining the opening is, for example, planar or non-planar, or protruding. When the surface defining the opening comprises an aperture of an on-chip device, preferably, the surface topography at the aperture is also protruded such that the opening is in a different plane from the remainder of the insulating surface forming the device, and preferably, is higher than the remainder of the insulating surface by at least about 1 μm-1000 μm., and preferably, by at least about 1-100 μm. Generally, the size of the protrusion is selected to be large enough to create stress on a cell surface.

Alternatively, or additionally, the surface is treated so as to render at least the cell membrane-contacting portions of the surface hydrophilic, e.g., such as by an RCA cleaning method, or by flame-treating, or by chemical treatment, as described above.

Alternatively, or additionally, surface features at the opening may be modified to enhance the formation of a high electrical resistance seal. For example, cells have been shown to arrange, interact with, and react to, nanoscale structures such as reeves, columns, rods, and protrusions in surfaces and these interactions have been demonstrated to be important for cell motility, positioning and ability to attach to surfaces. Thus, nanostructured surfaces are likely to be important in the sealing process and to provide stable seals for long-term recordings. Nanostructures can be generated on surfaces for separating a cell from an electrode compartment or channel using methods known in the art, such as by hard or soft lithography, vapor deposition, or by Atomic Force Microscopy (AFM).

In another preferred aspect, the patch clamp array device, the surface topography of the sensor chamber or channel itself is designed to maximize the seal between a cell membrane and the opening of the sensor chamber. In one aspect, the chamber comprises a non-planar surface feature that restricts the movement of the cell within the chamber and/or helps to position the cell relative to the surface defining the opening, to increase the electrical resistance of the seal between the cell and cell-contacting surface. For example, a pyramidal structure can be microfabricated at the base of the sensor chamber. In one aspect, the tip of the pyramidal structure is recessed so as to receive a cell.

A cell membrane is preferably placed in proximity to the surface comprising the opening. The addition of the cells to individual chambers of an array device, can be mediated by dispensing them, e.g., such as by using nQUAD aspirate dispensers. Other methods can used to position a cell such as electrophoresis, suction, the use of voltage pulses, and the like.

In one aspect, pressure-driven flow is used to manipulate the movement of cells from microfluidic channels in a substrate to an appropriate sensor chamber or channel. Routing of cells can be affected by blocking a branch of a channel in a substrate comprising a plurality of microchannels, using valves as are known in the art, thereby moving the cells along with bulk solution flow into another, selected channel.

Additionally, or alternatively, electroosmosis can be used to produce motion in a stream containing ions, e.g., such as buffer solution, by application of a voltage differential or charge gradient between two or more electrodes. Neutral (uncharged) cells can be carried by the stream. See, e.g., as described in U.S. Published Application No. 20020049389.

Dielectrophoresis is believed to produce movement of dielectric objects, which have no net charge, but have regions that are positively or negatively charged in relation to each other. Alternating, non-homogeneous electric fields in the presence of cells cause the cells to become electrically polarized and thus to experience dielectrophoretic forces. Depending on the dielectric polarizability of the particles and the suspending medium, dielectric particles will move either toward the regions of high field strength or low field strength. The polarizability of living cells depends on the type of cell and this may provide a basis for cell separation, e.g., by differential dielectrophoretic forces. See, e.g., as described in U.S. Published Application 20020058332.

Radiation pressure can also be used to deflect and move cells with focused beams of light such as lasers or optical tweezers.

In another aspect, the system is part of a cell-based biosensor such as is described in U.S. Provisional Application 60/356,377, filed Feb. 12, 2002, the entirety of which is incorporated by reference herein.

The exact geometry of the sensor chamber is not limiting, so long as it is able to support a cell or portion thereof, or a plurality of cells or portions thereof, in proximity to at least one electrode compartment or channel, such as a patch clamp micropipette or a channel in a microfluidic device or system. In this aspect, the chamber typically comprises a bath solution that is physiologically compatible with an intact cell. The at least one electrode compartment or channel comprises an electrolyte solution for maintaining suitable electrical communication between a cell membrane and an electrode within the electrode compartment or channel. The cell is separated from the electrode compartment or channel by a surface defining an opening through which the electrolyte solution can flow, electrically coupling the cell to the one or more electrodes in the electrode compartment or channel.

The cell can be moved in proximity to the electrode compartment or channel or electrode channel using fluid flow. Alternatively, or additionally, a cell can be moved using optical tweezers or by moving the electrode compartment or channel itself (e.g., through the used of a micropositioner, such as when the electrode compartment or channel comprises a patch clamp micropipette). The sensor chamber itself can be configured to include one or more electrical elements for creating an electrical field to aid in positioning cell(s) in proximity to an appropriate electrode compartment or channel, e.g., to create electroosmotic flow within the sensor chamber or to polarize a cell to facilitate its movement towards an electrode compartment or channel.

Fluid flow also can be used to increase the electrical resistance of a seal between a cell membrane and a surface defining an opening that separates the cell from an electrode compartment or channel. For example, a cell, loosely attached at the opening of the surface, can be placed in proximity the outlet of a fluid flow source providing a liquid stream. Or pressure may be used to increase the electrical resistance of a seal between a sensor and a surface defining an opening that separates While the cell is exposed to the flow, the area of cell membrane that contacts the surface defining the opening increases dramatically creating a stable seal.

Accordingly, in one aspect, a cell membrane is placed in sufficient proximity to a fluid stream to receive pressure from the stream. This pressure facilitates formation of or enhances, a seal between a cell membrane and the opening of the surface that separates the cell membrane from an electrode compartment or channel. The fluid stream may be provided to a chamber comprising a cell, such as an open volume chamber in a cell-based biosensor, as described above. In an on chip patch clamp device, the fluid stream may be provided to a cell through microfluidic channels microfabricated in the device using methods routine in the art.

Preferably, the fluid flow source provides a liquid stream with a fluid velocity ranging from 0.01 mm/s to 100 cm/s, preferably, 0.1 mm/s to 10 cm/s.

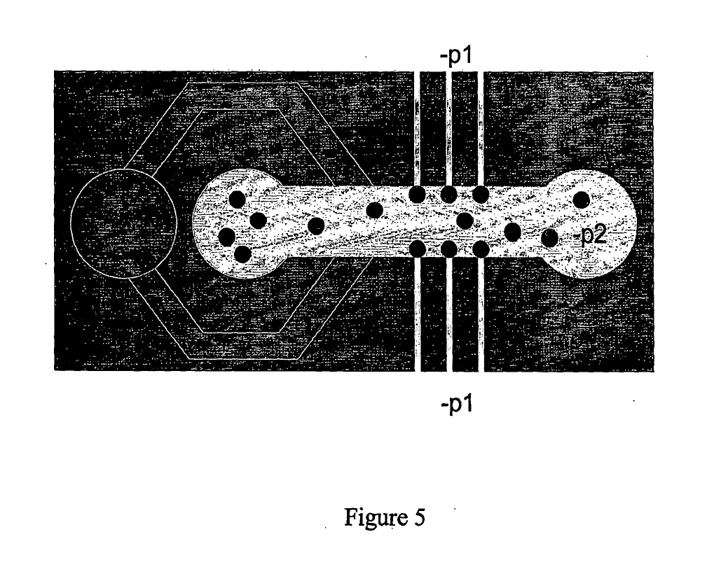

Accordingly, in one aspect, the invention provides, a cell-based biosensor having a fluid flow source comprising at least one outlet entering a chamber or reservoir for containing one or more cells. The fluid flow source can comprise at least one microchannel capable of providing a fluid stream to one or more cells (see, e.g., FIGS. 5-8). In another aspect, the fluid flow source comprises a plurality of outlets for providing a plurality of fluid streams to for example, selectively expose the cells or help position the cells at or near an opening in a surface, for example, an opening to a channel (e.g., a channel comprising an electrode. The plurality of outlets may lie in a single plane or in multiple planes, e.g., such as in the form of a stack of microchannels on a substrate. Multiple fluid flow sources can be provided as part of a single substrate providing fluid streams which flow in different directions, e.g., such as perpendicular to each other to enable a cell to be moved at an angle relative to the plane of the cell-contacting surface. Additional configurations of fluid flow sources are disclosed in U.S. Provisional Application 60/356,377, filed Feb. 12, 2002, the entirety of which is incorporated by reference herein.

In addition to the methods described above for forming high electrical resistance seals, a suction can be applied at the opening of the surface separating the cell from the electrode compartment or channel to enhance the electrical resistance of the seal. Alternatively, or additionally, one or several voltage pulses are applied at the opening to increase the electrical resistance of the seal (e.g., using the internal electrode of a channel or the one or more electrodes of a patch clamp array).

Alternatively, the sequence of events can be the following:

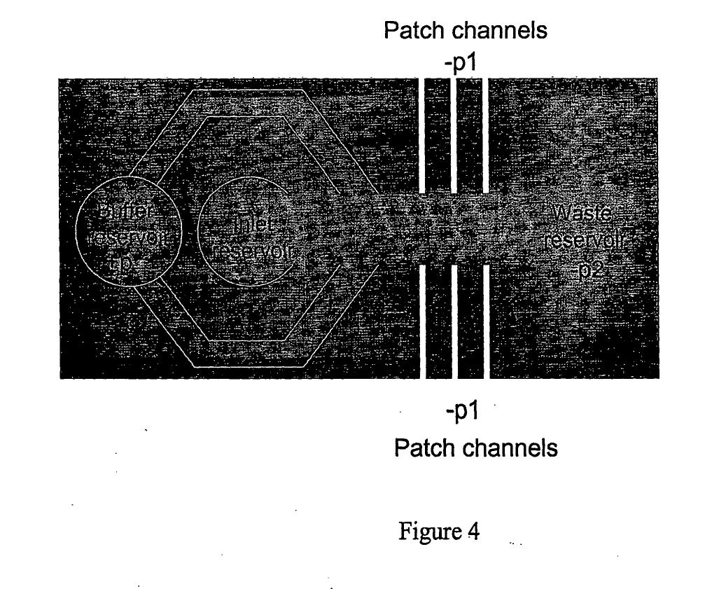

In one embodiment, the method of operation of the six-patch site unit is shown in FIG. 4. The buffer reservoir 200, buffer channels 120, inlet reservoir 210, inlet channel 110, delivery channel 100, and waste reservoir 220 are filled with buffer solution.

Other steps in the method of use of an exemplary system is shown in FIG. 5. For example, cells are added to the inlet reservoir 210. Negative pressure −p2 is applied at the waste reservoir 220 using a pressure source. As cells migrate down the delivery channel 100, negative pressure −p1 is applied at the electrode channels and transmitted through the patch site channels 130 to the patch sites (openings of the channels) 150, immobilizing individual cells at the six patch sites (channels openings) 150.

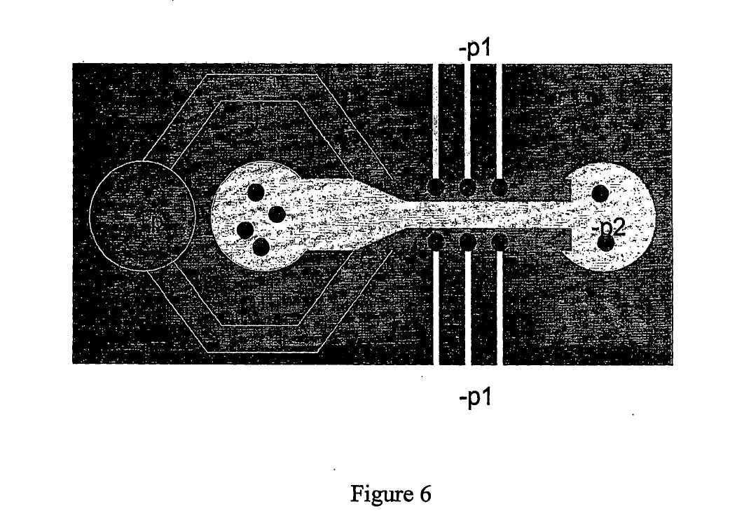

Another step is depicted in FIG. 6. For example, positive pressure may be applied to the buffer reservoir 200, creating a laminar flow 125 of buffer fluid along each side of the delivery channel, thereby bathing the patch cells with the buffer fluid. Negative pressure continues to be applied at the waste reservoir 220 to empty the inlet reservoir 210. The pressure may be switched so that the fluid stream being exposed to the cells changes rapidly.

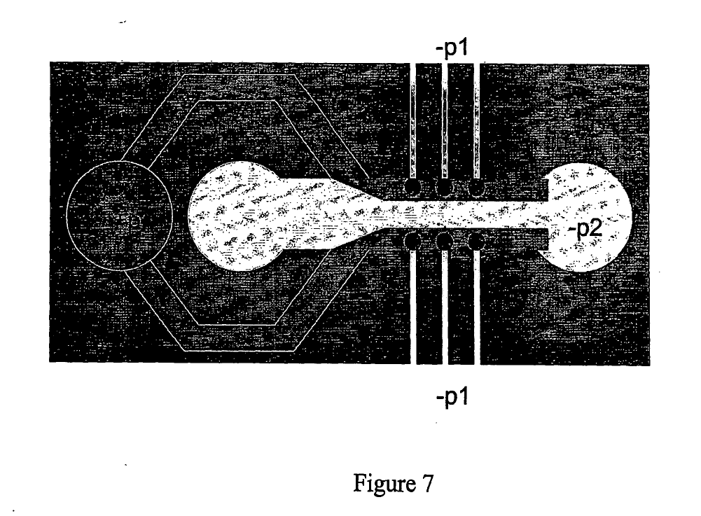

Yet another method step in the operation of the six-patch site unit is depicted in FIG. 7. A substance (or ligand) is added to the inlet reservoir 210. While the patch cells continue to be bathed and protected by the laminar flow 125 of buffer along each side wall of the delivery channel 100, negative pressure −p2 applied at the waste reservoir 220 pumps the substance through the main part 115 of the delivery channel 100.

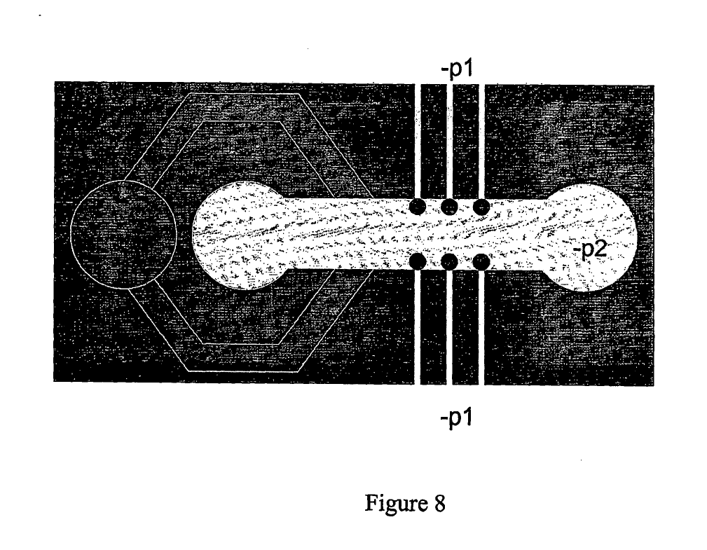

FIG. 8 depicts the removal of positive pressure from the buffer reservoir 200. Laminar flow 125 over the cell patch sites is switched. The patch sites 150 are now exposed to the substance flowed in from the inlet reservoir 210 via the inlet channel 110. Measurements are taken of the sensor at any time during the process, either continuously or intermittently.

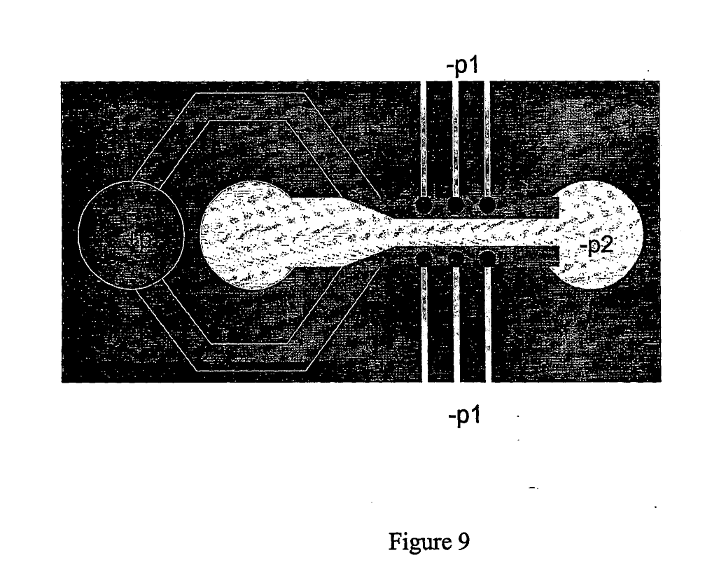

FIG. 9 depicts positive pressure being re-applied in the buffer reservoir 200, and buffer flow resumes via the bifurcated channels 120, re-establishing a laminar flow 125 along the side walls of the delivery channel 100. With the cells now bathed and protected by buffer, the substance is washed out of the inlet reservoir 210, the inlet channel 110 and the delivery channel 100. A new substance or a different concentration of substance may now be loaded into the inlet reservoir, and the cycle is repeated for a new set of measurements.

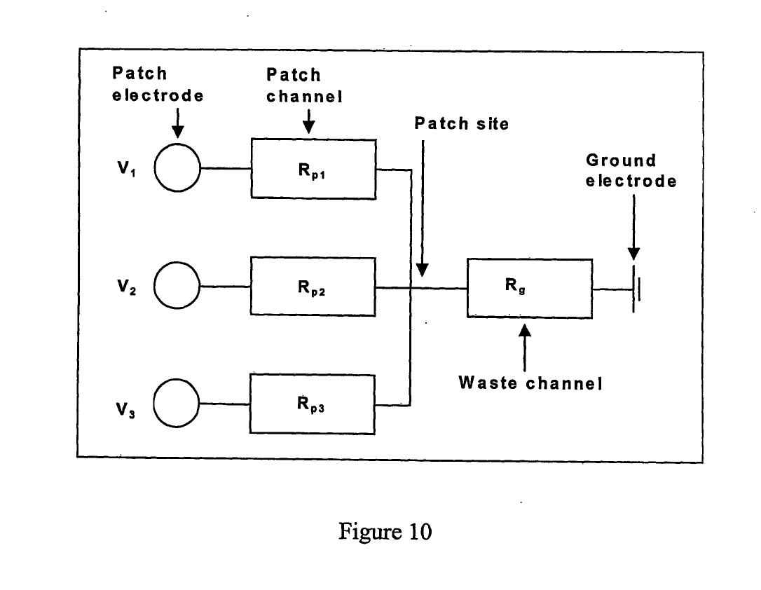

FIG. 10 depicts the measurement circuitry associated with one side of the six-patch site unit. Individual patch electrodes V1-V3 are positioned in the patch reservoirs, which communicate with the individual patch sites. The resistance associated with each patch channel creates a voltage drop at the corresponding electrodes V1-V3. The ground electrode is placed within the waste reservoir, and the waste channel resistance provides a common voltage drop for all of the electrodes.

Another embodiment comprises a surface comprising an opening for separating a cell membrane from an electrode compartment or channel positioned close to a cell membrane by either moving the cell membrane via fluid flow. A small suction and/or one to several voltage pulses are applied at the opening.

A cell membrane, loosely held at the surface (e.g., less than 0.01 μm from the surface), is placed in proximity to the outlet of a fluid flow source that provides a liquid stream. While the cell membrane is exposed to the flow stream, the surface area of the membrane in contact with the surface increases dramatically, creating a stable seal. After a predetermined time or when a satisfactory electrical reading of resistance is reached, the cell is taken out of the flow stream, whereupon more suction or more voltages are applied to at the surface until a suitable recording configuration is achieved, e.g., one which does not vary significantly over multiple sequential readings.

In one embodiment, a cell is guided to an electrode channel defining an opening in the sensor chamber for separating a cell from an electrode channel. A flow stream normal to the generally planar portion of the insulating surface is provided to exert a pushing force on the cell. Where the device comprises multiple electrode channels, cells can further be automatically positioned at a plurality of openings to such compartments by moving cells in a stream at an angle greater than or lesser than 90° to the base surface or perpendicular to this plane and/or by applying pressure to the channels. A suction pressure and/or voltage may be applied at the openings such that the cells are attracted or drawn to the openings of respective electrode channels. Alternatively or in addition to, dielectrophoresis can be used as known in the art or other alternating current (ac) methods, as described above.

The systems described above can be used in any method that generally comprises determining the electrical properties of one or more cell membranes. Suitable cells or portions thereof for use in the method include, but are not limited to, bacterial, yeast, insect, and cells. For example, Bacillus spp., Escherichia coli, Streptococcus spp., Streptomyces spp., Pseudomonas spp., can be used. Yeast cells such as Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytic, as well as other lower eukarotes, also can be used.

Insect cell lines may also be used, including, but not limited to, Aedes aegypti, Autographa californica, Bombyx mori, Drosophila melanogaster, Spodoptera frugiperda, and Trichoplusia ni.

Mammalian cell lines include, but are not limited to immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Madin-Darby bovine kidney (“MDBK”) cells NIH/3T3, 293 cells (ATCC #CRL 1573), COS-7, 293, BHK, CHO, TM4, CV1, VERO-76, HELA, MDCK, BRL 3A, W138, Hep G2, MMT 060562, TRI cells, as well as others. A well-known example of an avian cell line is the chicken B cell line “DT-40”.

Specific animal cells include, but are not limited to, leukemia L1210 cells (In Modern Pharmacology, pp. 1121-1129 (1978)); guinea pig heart cells (Journal of Physiology 397:237-258 (1988); starfish egg cells (The Journal of General Physiology 70:269-281 (1977) and denervated frog muscle fibers (Neher et al., Nature 260 (Apr. 29, 1976).

Cells analyzed using the systems and methods of the invention include cells that have been transfected to express recombinant gene products. For example, cells can be engineered to express particular ion channels by transfecting such cells with appropriate cDNAs (see, e.g., U.S. Pat. No. 5,670,335).

As discussed above, artificial cells or vesicles also can be used with/or without recombinantly made proteins inserted into the membranes of such cells. See, e.g., U.S. Pat. No. 5,795,782 and U.S. Pat. No. 6,022,720.

Accordingly, in one aspect, a system comprising a surface defining an opening which separates a cell membrane from an electrode compartment or channel is provided and the cell membrane is placed in sufficient proximity to the opening and exposed to conditions in which a high electrically resistant seal forms with between the cell membrane and the surface (e.g., a resistance of at least about 1 Gohm). In one aspect, the surface defining the opening is, for example, planar, non-planar, or protruded. Alternatively, or additionally, the surface is hydrophilic. In one aspect, fluid flow and/or pressure is used to position the cell membrane in seal forming proximity to the surface defining the opening. Alternatively, or additionally, fluid flow is used to maximize the electrical resistance of a seal already formed. Preferably, at least one measurement of an electrical property of the cell membrane is obtained such as a voltage or current across the cell membrane. More preferably, electrical propert(ies) are measured as the cell is responding to, or after a cell has responded to, a condition and/or agent in a bath solution surrounding the cell.

Examples of agents, include, but are not limited to, proteins, DNA, RNA, PNA, receptor agonists, receptor antagonists, neurotransmitter, neurotransmitter analogues, enzyme inhibitors, ion channel modulators, G-protein coupled receptor modulators, transport inhibitors, hormones, peptides, toxins, antibodies, pharmaceutical agents, chemicals, purinergics, cholinergics, serotonergics, dopaminergics, anesthetics, benzodiazepines, barbiturates, steroids, alcohols, metal cations, cannabinoids, cholecystokinins, cytokines, excitatory amino acids, GABAergics, gangliosides, histaminergics, melatonins, neuropeptides, neurotoxins, endothelins, NO compounds, opioids, sigma receptor ligands, somatostatins, tachykinins, angiotensins, bombesins, bradykinins, prostaglandins and combinations thereof.

A search for genes encoding ion channels or transporter proteins can be carried out by parallel transfection of cells with genes to be tested, followed by screening for ionic currents as described herein.

The systems described herein may also be useful for screening compound libraries, to characterizations the pharmacological properties of compounds, and to obtain dose-response data.

Examples of agents that may be used for the apparatus and methods of the invention include drugs, receptor agonists, receptor antagonists, neurotransmitter, neurotransmitter analogues, enzyme inhibitors, ion channel modulators, G-protein coupled receptor modulators, transport inhibitors, hormones, peptides, toxins, antibodies, pharmaceutical agents, chemicals and combinations of these agents. Specific agents which may be used for the systems and methods of the invention include purinergics, cholinergics, serotonergics, dopaminergics, anesthetics, benzodiazepines, barbiturates, steroids, alcohols, metal cations, cannabinoids, cholecystokinins, cytokines, excitatory amino acids, GABAergics, gangliosides, histaminergics, melatonins, neuropeptides, neurotoxins, endothelins, NO compounds; opioids, sigma receptor ligands, somatostatins, tachykinins, angiotensins, bombesins, bradykinins, prostaglandins and combinations of these agents.

Rapid Alterations of the Solution Environment Around a Sensor

Described herein is the use of two-dimensional (2D) and three-dimensional (3D) networks of microfabricated channels for the complex manipulation of compounds or reagents contained in the fluid in a way that permits repeated and rapid delivery of different solutions to the sensor in the sensor chamber. For example, the microfluidics used with the system enables the system to programmably deliver a ligand to a cell-based biosensor comprising a receptor. This enables the system to be used for HTS screening of samples (e.g., such as compound libraries) to monitor the effects of compounds on the responses of the biosensor. In one aspect, electrical properties of a cell-based biosensor are monitored using voltage clamp or patch clamp techniques.

Because the system provides a mechanism for changing solutions rapidly around a sensor, the system can be used to flush a cell-based biosensor with buffer after exposure to a sample compound, enabling a receptor or ion channel that is part of the biosensor to be resensitized prior to exposure to the next compound. Thus, the system can provide a periodically resensitized receptor for exposure to potential modulators of receptor function (e.g., such as agonists or antagonists). For receptors that do not desensitise, the system is still advantageous for providing pulsed delivery of buffer to a receptor, e.g., to remove unbound ligand from the receptor, to enhance the specificity and/or decrease background of a response.

The geometry of different network structures of microchannels is designed to exploit the unique characteristic of fluid behavior in micro-dimensions. Three exemplary designs are described below.

The system has the ability to flow different stream across one or more sensors rapidly. For example, the system also can sweep different fluid streams across a stationary sensor by varying pressure drops across the substrate, for example in the same channel, e.g., laminar collimated fluid streams. The system requires small sample volumes (nLs to μLs) and can be easily automated and programmed for HTS applications.

The Rapid Transport of Sensors Across Different Streams of Fluids

Adjacent fluid streams (laminar and collimated) flowing through a substrate according to the invention have a low Reynold's number and undergo minimal mixing by diffusion. The fluid streams may be switched while flowing through the sensor channel to rapidly and selectively expose a sensor to a different stream. For example, a small molecule with a diffusion coefficient of about 5×10⁻⁶ cm²/s would take approximately 0.1 seconds to diffuse 10 μm, but 10 s to diffuse 100 μm, owing to the square dependence of distance on diffusion time (x²=2Dt, where D is the diffusion coefficient). Similarly, for typical proteins having D ˜10⁻⁶ cm²/s, it will take 0.5 second to diffuse 10 μm and 50 seconds for 100 μm. See, for example, U.S. application Ser. No. 10/345,107 and U.S. application Ser. No. 10/645,834., which are hereby incorporated by reference in their entirety.

At the flow rates for use with patch clamp measurements and at a cell-to-outlet distance of about 20 μm or less, the different fluid streams are essentially distinct and separate and are undisturbed by the presence of a patch-clamped cell. Even at much lower flow rates (e.g., <100 μm/s) that may be used with patch clamp measurements, different fluid streams are still well separated. This observed behavior (e.g., collimation of fluid streams) of fluid flow facilitates HTS applications which require relatively rapid translation of patched cells with respect to different fluid streams. For a stationary cell, such as a patched cell or an immobilized cell in channel under fluorescence observation, this rapid translation of patched cell with respect to the different fluid stream can be achieved by rapid variation of the relative pressure drops across the different streams.

Patch Clamp or Cell Holding for Measurement Under Fluid Flow

The ability to rapidly switch patch-clamped cells across interdigitated streams of receptor modulators (agonists or antagonists) and buffer depends on the mechanical stability of the patched cell under the required flow conditions as well as scan speeds. Here, the stability of the “giga seal” and ion-channel activities of patch-clamped cells under a range of flow conditions is described.

The effects of liquid flow on a patch-clamped cell arise from the force (Stokes drag) exerted by the flow on the cell. This Stokes drag can be calculated from the following equation:

Force=(frictional coefficient)×(velocity of the flow)

Where the frictional coefficient (f) can be calculated from:

f=6πrμ

where r is the radius of the cell and μ is the viscosity of the solution. This relationship is valid for low Reynold's number flow and for particles that are spherical. Both conditions are adequately met in the methods and devices utilized in connection with the present invention.

For water at room temperature, μ is ˜1 centipoise (1 centipoise=0.01 g/[cm s]) and for a typical mammalian cell, r=5 μm. Using these values and for flow rates of 1 mm/s, Force=9.4×10⁻¹¹ N or 94 picoNewtons. Since force is linearly proportional to the flow rate, at 0.1 mm/s, Force is 9.4 picoN. To put this number in perspective, micropipettes can routinely exert nano- and micro-Newtons on a small particle such as a cell. In addition to the force that arises owing to the drag on the cell from fluid flow, the scanning of the cell at a certain velocity exerts a similar drag force in the direction of cell translation, which is typically orthogonal to the direction of fluid flow. Scanning of a cell at 1 mm/s under no flow typically has the same effect as keeping the cell stationary while flowing the fluid at the same rate.

For applications that require extremely high flow rates in which cell dislodgement may become an issue, patch-clamped cell(s) may be put into a recessed region or well in the sensor chamber that matches the dimension of the cell. The well may be a well at the opening of the electrode channel. This design will permit the use of high flow rates while preventing cell dislodgement because the flow profile in a channel or chamber is parabolic, owing to no-slip boundary conditions at the interface of a fluid and a solid surface (e.g., the velocity at the interface of the fluid and the solid surface is zero). By placing cell(s) in well(s) having similar dimensions as the cell, the cell is essentially “shielded” from the high velocity flow region that is located away from the well and the solid surface. Therefore, although the average flow rate and the flow velocity away from the solid surface can be extremely high, the flow velocity near the well in which the patched cell is placed can be very small. By using this strategy, very high average flow rates can be used.

Because the aqueous solutions flowing through the channels are non-compressible (unlike air), the width and placement of each fluid stream depends on the relative flow rate through the microchannel. Therefore, fluid streams from the microchannels also can be made to move and translate by varying the flow rate through each channel. This is most easily achieved by controlling the pressure drops across each channel or by changing the resistance of each channel. The ability to move fluid streams by pressure variations (or other means) is particular useful in applications in which the sensor(s) are cell-based and are immobilized on the chip, such that such that mechanical movements of the cell(s) relative to the chip are not possible. The pressure and resistances of each channel can be programmed, using the system processor. Parameters which can be programmed include, but are not limited to, linear changes in the pressure and resistance of each channel, stepwise or constantly variable changes in the pressure and resistance of each channel, and the sequence of changes among the different channels. In addition, pressure and resistance changes can be based on real-time feedback signals, and these signals may be processed and computed prior to outputting new pressure and resistance parameters.

A sensor may comprise a receptor/ion channel which does not desensitize, eliminating the need to resensitize the receptor. However, the system may still be used to provide pulsed delivery of buffer, for example, to wash a cell free of unbound compounds. In this scenario, the scan rate can be adjusted based on “noise” observed in the response. For example, the solution change rate can be adjusted to achieve a linear dose-response over certain concentrations of sample compound.

A ligand also may irreversibly block a sensor, rendering it unresponsive to other ligands in other fluid streams. In this case, pulsing with buffer will have no effect. It is straightforward to ascertain whether the cell is inactivated by introducing compounds of known effect periodically to the cell and verifying whether an appropriate response is obtained. Preferably, the system is able to sense a lack of response by a sensor as it is exposed to a selected number of sample fluid streams. For example, the system can provide a feedback signal when no response is observed in patch clamp recordings over as a sensor is exposed to a selected number of consecutive fluid streams.

Alternatively, or additionally, devices can be provided in the sensor chamber to monitor sensor function. In one aspect, an optical sensor is provided in communication with the sensor chamber for monitoring the viability of a cell-based biosensor. For example, spectroscopic changes associated with cell death (e.g., such as from chromatin condensation) may be observed, or the uptake of a dye by a dead or dying cell can be monitored.

Fluorescent measurements may also be used to measure various aspects of binding, movement across the membrane, cellular localization, calcium increases, and the like.

In one aspect, the system executes certain program instructions when a selected number of exposing intervals in which no sensor signal has been received have gone by. For example, the system can vary pressure at particular channels to stop flow in those channels, thereby minimizing sample waste. In another aspect, in response to an absence of a response signal from a sensor over a threshold period, one or more replacement biosensors are delivered to the sensor chamber (e.g., from the cell treatment chambers described above).

If a sensor is translated at a constant speed compared to flow rate from channel outlets (e.g., mm/s), then the exposing rate (e.g., compounds screened per second) for channels having a width and spacing of about 10 μm will be approximately 25 Hz. Using about 100 μm wide channels with channel intervals of about 10 μm, the exposing rate will be about 4.5 Hz. If the translation speed is increased, the exposing range may be in the range of hundreds of Hz. For some applications, e.g., where the sensors comprise rapidly desensitizing ion channels, fluidic channels with narrow outlets are preferred as these can provide sharp concentration profile over short periods of time. Preferably, such channels range from about 1 μm to about 100 μm in width.

Exposing rates can be uniform or non-uniform. For example, exposing rates across channels providing sample streams (e.g., providing agonists) can differ from exposing rates across channels providing buffer streams. Variable exposing rates can be based on preprogramming or on feedback signals from the sensor measurements, e.g., such as from patch clamp measurements. The actual scan rate will vary depending on the exact screening system, but a typical linear scan rate will range from between about 100 μm/s to hundreds of mm/s for a sensor comprising a mammalian cell having a diameter of about 10 μm.

Cycles of Rapid Delivery

Another feature of the system according to the invention is that fluid can be rapidly delivered through the channels into the sensor chamber, enabling compounds to be introduced into the microenvironment of a sensor and withdrawn from that microenvironment rapidly.

Fluid flows inside micron-sized channels are laminar and reversible, a property that can be gauged by a dimensionless number, called the Reynold's number (Re): For example, typically, fluid flow having a low Re number is reversible, while at high Re numbers, fluid flow becomes turbulent and irreversible. The transition between laminar reversible flow and turbulent flow appears to occur at a Re number of about 2000, an estimation based on flow through a smooth circular channel (e.g., approximating flow through a microchannel). Even at high flow rates (m/s), Re for channels measuring a few microns in width is ˜<10. This means that fluid flow in micron-sized channels fall well within the laminar reversible regime. The key feature of fluidic behaviour exploited herein is the reversibility of fluid flow.

In one aspect, positive pressure is applied at a microchannel to introduce a compound or drug into the sensor chamber housing the biosensor, preferably a patch-clamped cell. After a suitable incubation time to allow interaction between the compound/drug and the biosensor, a negative pressure is applied to withdraw the compound/drug from the chamber. Because fluid flow is completely reversible and also because diffusion is negligible under conditions used (e.g., relatively fast flow), the drug is completely withdrawn from the chamber back into the microchannel from which it came. In this way, each compound delivered onto the cell to screen for potential interactions, can be subsequently withdrawn from the cell so the cell is again bathed in buffer, re-sensitized, and ready for interaction with the next compound delivered via a different microchannel.

Rapid Exchange of Fluids

This design relies on the fact that solutions contained in the microchannels and sensor chamber (and/or cell treatment chambers) can be rapidly and efficiently replaced and exchanged. Rapid solution exchange can be achieved using a variety of different microchannel network geometries. In one aspect, a plurality of microchannels converge or feed into the sensor chamber, while in another aspect, a plurality of microchannels converge into a single channel which itself converges into the sensor chamber. The plurality of microchannels can comprise interdigitating channels for sample and buffer delivery respectively. In a preferred aspect, the design is integrated with a patch clamp system. Three exemplary constructions are described below.

The dimensions of the microchannels in the system (width and thickness) (for both sample delivery and buffer delivery) can be highly variable, with typical dimensions ranging from about 1-100 μm, and preferably from about 10-90 μm. Flow rate also may be varied with preferred flow rates ranging from μm/s to cm/s.

Pressure is isotropic, therefore, upon application of a positive or negative pressure, fluids will flow along any pressure drop without preference to any particular direction. Therefore, preferably, passive one-way valves are integrated at the junction between sample delivery microchannels and the main buffer channel. The purpose of these integrated one-way valves is to prevent any flow from the main buffer channel into each of the sample delivery microchannels upon application of a positive pressure to the buffer reservoir, while allowing flow from each of the sample delivery microchannels into the main buffer channels when positive pressure is applied to reservoirs providing sample to these microchannels. There are numerous suitable designs for microfluidic valves as well as pumping mechanisms.

Although the discussion below emphasizes pressure driven flow owing to its simplicity of implementation, a number of appropriate means can be designed for transporting liquids in microchannels, including but not limited to, pressure-driven flow, electro-osmotic flow, surface-tension driven flow, moving-wall driven flow, thermo-gradient driven flow, ultrasound-induced flow, and shear-driven flow. These techniques are known in the art.

Valving and Pumping

Scheme 1: Using Septum to Address Individual Microchannels

In this scheme, the reservoirs that connect to each of the microchannels are sealed by a septum, for example, using polydimethyl siloxane (PDMS) or a double-sided adhesive for sealing or another suitable material as is known in the art. Because the septum forms an airtight seal, application of a positive pressure (e.g., with air or nitrogen) via a needle or a tube inserted through the septum will cause fluid to flow down the microchannel onto one or more sensors in a sensor chamber (e.g., to the center of a spokes-wheel where radial microchannels converge). Application of a negative pressure with a small suction through the needle or tubing inserted through the septum will cause fluid to be withdrawn in the opposite direction (e.g., from the chamber at the center of the spokes-wheel to the reservoir feeding into the microchannel).

An array of such needle-septum arrangements allows each reservoir to be individually addressed, and therefore, each microchannel. The use of this scheme permits the simultaneous and sequential pumping and valving of the fluids contained within each of the microchannels. By exercising precise control over positive and negative pressure applied to each of the microchannels, controlled fluid flow and compound delivery onto the one or more sensors can be achieved. For designs that do not require individual addressing of the microchannels (e.g., design 1—the rapid transport of patched cells across different streams of fluids), a single or a few septa with a single or a few pressure control devices will suffice.

Scheme 2: Control of Fluid Flow With External Valves

In this configuration, compounds from each of the wells of an array well plate are introduced through external tubings or capillaries which are connected to corresponding microchannels. External valves attached to these external tubings or capillaries can be used to control fluid flow. A number of suitable external valves exist, including ones actuated manually, mechanically, electronically, pneumatically, magnetically, fluidically, or by chemical means (e.g., hydrogels).

Scheme 3: Control of Fluid Flow With Internal Valves

Rather than controlling fluid flow with external valves, there are also a number of chip-based valves that can be used. These chip-based valves can be based on some of the same principles used for the external valves, or can be completely different, such as ball valves, bubble valves, electrokinetic valves, diaphragm valves, and one-shot valves. The advantage of using chip-based valves is that they are inherently suited for integration with microfluidic systems. Of particular relevance are passive one-way valves, which are preferred for implementing some of the designs mentioned in above (e.g., such as the branched channel format).

Other suitable geometries may be integrated with any of the above systems. In one aspect, at least one channel of a microfluidic system described above is a mixing channel which receives two or more separate streams of fluid from two or more other channels. The mixing channel can be used to combine the separate streams in a single channel. Such a configuration can be used to establish a concentration gradient of a substance provided in different concentrations in the two or more separate streams as is described in WO 02/22264.

### Detection

The system can be used to monitor cellular responses by measuring changes in electrical properties of cells. In one aspect, the sensor chamber of the chip comprises a cell-based biosensor and the system comprises a detector for monitoring the response of the biosensor to solution flow from the channels. One response which can be monitored is a change in an electrical property of the biosensor in response to gating of an ion channel. For example, a change in current flowing across the membrane of the biosensor can be measured using a voltage clamp technique. Currents can be in the range of a few picoampere (pA) (e.g., for single ion-channel openings) to several μA (for cell membranes of larger cells such as Xenopus oocytes).

Among voltage clamp techniques, patch clamp is most suitable for measuring currents in the pA range (see e.g. Neher and Sakmann, 1976, supra; Hamill, et al., 1981, supra, Sakmann and Neher, 1983, supra). The low noise property of patch clamp is achieved by tightly sealing a glass microelectrode or patch clamp pipette onto the plasma membrane of an intact cell thereby producing an isolated patch. The resistance between the pipette and the plasma membrane is critical to minimize background noise and should be in excess of 10⁹ ohm to form a “giga seal”. The exact mechanism behind the formation of the “giga seal” is debated, but it has been suggested that various interactions such as salt-bridges, electrostatic interactions, and van der Waal forces mediate the interaction between the glass surface of the pipette and the hydrophilic heads in the lipid layer of the cell membrane (see, e.g., Corey and Stevens, 1983, In Single-Channel Recording, pp. 53-68, Eds. B. Sakmann and E. Neher. New York and London, Plenum Press). Variations of patch clamp techniques can be utilized such as whole-cell recording, inside-out recording, outside-out recording, and perforated patch recording as are known in the art.

In whole-cell recording, the cell membrane covering the electrode tip is ruptured by suction in order to establish an electrical connection (and a chemical pathway) between the cell interior and the electrode solution. Because electrode solution is in great excess compared to the amount of cytosol in the cell (about 10 μl vs. about 1 μl), changing ionic species in the electrode solution will create concentration gradients across the cell membrane, providing a means to control the direction and magnitude of the transmembrane ionic flow for a given receptor/ion-channel complex.

In inside-out and outside-out patch clamp configurations, the cytosolic environment is lost by excision of a membrane patch from the entire cell (see, e.g., Neher and Sakmann, 1976, supra; Sakmann and Neher, 1983, supra). The inside-out configuration allows exposure of the cytosolic side of the membrane to solution in the chamber. It is therefore a method of choice for studying gating properties of second-messenger activated ion-channels at the single-channel level.

Low noise levels provide better signal-to-noise ratios where modulators (e.g., such as agonists or antagonists). Under optimal conditions, single-channel currents in the higher femto-ampere (10⁻¹⁵ A) range can be resolved. Strategies to decrease noise (e.g., such as caused by a bad seal between the electrode and the cell) to facilitate formation of GΩ-seals include, but are not limited to, fire polishing of the glass electrode or treating the surface the glass electrode using agents such as sigmacote. Dielectric noise and capacitive-resistive charging noise also can be decreased by selecting an expedient electrode/pipette geometry, using quartz glass, and by coating of the glass surface of the pipette with Sylgard® (silicone, PDMS) in order to insulate the pipette tip as much as possible.

One frequently used modification of the whole-cell configuration, the perforated patch mode, also can be used (see, e.g., as described in Pusch and Neher, 1988, supra). In this technique, holes are selectively made in the cell membrane using a pore-building protein, such as amphotericin or nystatin (see, e.g., Akaike et al., 1994, Jpn. J. Physiol. 11: 433-473; Falke, et al., 1989, FEBS Lett. 251: 167; Bolard, et al., 1991, Biochemistry 30: 5707-5715) to create increased conductivity across the patched cell membrane without the loss of intracellular signalling molecules.

In addition to measuring ion currents across ion channels at constant membrane potential, the patch clamp technique can be used to measure membrane voltage at a known constant or time-varying current. This patch clamp configuration, referred to as “current clamp”, measures the change in membrane potential caused by activation of ligand-gated ion-channels or by voltage-gated ion channels and is particularly suited for creating a biosensor which can be used to monitor the effects of agents (e.g., drugs) on action potentials (e.g., frequency, duration, and amplitude). This technique also can be used to study the effect of an agent to study an agent's impact on the excitability of a nerve cell. Therefore, in one aspect, the system is used to monitor the modulation of the voltage threshold (e.g., hyperpolarizing or depolarizing) of a cell-based biosensor in a current clamp mode when an action potential is triggered.

In another aspect, the system is used to monitor capacitance changes in cell membranes by providing a cell-based biosensor in the open volume reservoir and measuring impedance of the membrane across the membrane of the biosensor in an AC mode. For example, the system can be used to monitor the effect of agents on the release of vesicles from a cell (e.g., exocytosis) and/or on the uptake of vesicles by a cell (e.g., endocytosis).

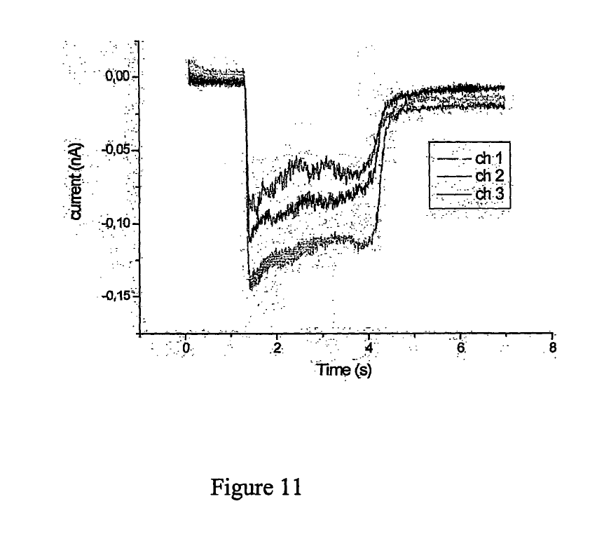

In one embodiment electrophysiological patch clamp recordings is shown in FIG. 11. The recordings were performed on WSS-1 cells expressing GABAA ion channel, immobilized and patch-clamped in a Nanoflow unit cell. The agonist; 500 μM GABA was applied between approximately 1.3 s and 4 s. Before and after GABA application, the cells were rinsed with extracellular buffer. The data indicates an average full solution exchange time of 55 ms for the three cells when switching on the agonist. The slower response of the switch back to buffer is a combination of slower switching due to diffusion in the liquid-liquid interface combined with physiological rinsing effects. External pumps and fluid control equipments are placed adjacent to a standard microscope. The entire integrated system preferably is computer-controlled and automated. The different components of the system may be controlled separately using separate controllers and separate software, but most preferably these components are all controlled by a single system processor as described above.

Besides electrophysiological measurements, another common and useful mode of detection is via fluorescence. For example, the cells may be loaded with fluorogenic dyes that indicate the concentration of calcium (e.g., fluoresces and emit light in the presence of calcium) or with potential sensitive dyes that report the cell membrane potential. Activation or inactivation of the cells will result in changes in the fluorescence optical signal, which can be easily detected with a photon detector.

Various supporting solutions can be adapted for use in sensor chamber. The type of solution will depend on the sensor and compounds being evaluated. For example, a sensor solution can be a recording solution used for traditional patch clamp analysis of an ion channel. In general, the exact composition of a solution for patch clamp recording will vary depending on the type of channel being evaluated (see, e.g., U.S. Pat. No. 6,333,337, for potassium channels; U.S. Pat. No. 6,323,191, for Cl⁻ channels, and PCT/US99/02008, for sodium channels); such solutions are well known in the art.

In one aspect of the invention, patch clamp recording is automated and controlled by the system processor. For example, the system processor may direct the solution flow, the solute concentration, the exposure to solutions, and/or the pressure being applied through the patch channel and/or the chamber or other channels and chambers. In one aspect, acquisition and analysis of patch clamp data, followed by a feedback control to vary microfluidic settings (e.g., pressure, valves and switches) and to control exposing parameters (e.g., speed and trajectory of exposing, pressure drops across channels), is implemented by the system processor.

Herein is described a method in which receptor proteins are prepared in discrete kinetic states characterized by having different response functions, dynamic range EC₅₀ and Hill slope. The present invention describes how accumulation of receptors in bound non-active states such as desensitisazed states and the dynamics between these states of the receptors essentially can be used as a molecular-level memory used in the construction of logic bio-devises and as well as in silica made in neuromorphic very large scare integration (VLSI) circuitry. Furthermore, the invention comprises a method for characterization and validation of receptor modulators such as drugs and pharmaceutically active substances by the fact that the response function, dynamic range, and the tuning of sensitivity in receptor proteins is altered by antagonist concentration and exposure time. The finding that competitive antagonist eradicates some of the differentiation in the response behavior may as well be the cause of some of the side effect for drugs acting on the GABAergic system.

### Methods of Using The System

The invention exploits the potential for using microfluidic systems to control the delivery of a large number of different biologically active molecules and compounds (e.g., candidate drugs) to a sensor comprising a target molecule. Suitable molecules/compounds which can be evaluated include, but are not limited to, drugs; irritants; toxins; proteins; polypeptides; peptides; amino acids; analogs and modified forms of proteins; polypeptides, peptides, and amino acids; antibodies and analogs thereof; immunological agents (e.g., such as antigens and analogs thereof, haptens, pyrogens, and the like); cells (e.g., such as eukaryotic cells, prokaryotic cells, infected cells, transfected cells, recombinant cells, bacteria, yeast, gametes) and portions thereof (e.g., cell nuclei, organelles, secretogogues; portions of cell membranes); viruses; receptors; modulators of receptors (e.g., agonists, antagonists, and the like); enzymes; enzyme modulators (e.g., such as inhibitors, cofactors, and the like); enzyme substrates; hormones; metabolites and analogs thereof; nucleic acids (e.g., such as oligonucleotides; polynucleotides; fibrinotides; genes or fragments, including regulatory sequences, and/or introns, and/or coding regions; allelic variants; RNA; antisense molecules, ribozymes, nucleotides, aptamers), including analogs and modified forms thereof, chemical and biological warfare agents; metal clusters; and inorganic ions.

Combinations of two or more of any of these molecules also can be delivered, sequentially or simultaneously, to one or more sensors in the sensor chamber. Compounds also can be obtained from synthetic libraries from drug companies and other commercially available sources known in the art (e.g., including, but not limited, to the LeadQuest® library comprising greater than 80,000 compounds, available through http://www.tripos.com/compounds/; ChemRx Diversity Library, comprising 1000 to 5000 compounds per scaffold, available through http://www.chemrx.com; the Nanosyn Pharma library, available through Nanoscale Combinatorial Synthesis Inc., Menlo Park, Calif., and the like) or can be generated through combinatorial synthesis using methods well known in the art. In aspects in which molecules are delivered to cells, any of the molecules described above may be taken up by cells by transiently exposing the cells to an electric field (e.g., in a cell treatment chamber or in a sensor chamber which is adapted for electroporation) as described above.

Providing Periodically Resensitized Ion Channel Sensors

Binding a compound (such as an agonist or modulator or drug) to a broad range of ion channels not only evokes conformational changes in these channels, allowing a flux of ions across a cell membrane, but also causes the ion channel to desensitize, e.g., to reside in a long-lasting, ligand-bound, yet shut-off and non-conducting state (see, e.g., Jones and Westbrook, 1996, GL Trends Neurosci. 19: 96-101). Desensitization of many types of ion-channels usually occurs within a few milliseconds and is thought to be one of the mechanisms by which synaptic information in the central nervous system is processed and modified. Densitization also may serve as a negative feedback mechanism that prevents excitotoxic processes caused by excessive activation of ion channels by neurotransmitters or other neuromodulators (see, e.g., Nahum-Levy, et al., 2000, Biophys J. 80: 2152-2166; Swope, et al., 1999, Adv. Second Messenger Phosphoprotein. Res. 33: 49-78).

In one aspect, to achieve high screening rates in, for example, HTS applications, patch-clamped cell(s) in the sensor chamber are exposed to different fluid streams in rapid succession. To achieve rapid resensitizaton of ion channels and receptors, delivering samples comprising suspected modulators, agonists, or drugs of receptor/ion channels are switched via pressure changes within the sensor chamber to deliver buffer for resensitization of the receptor/ion channels (e.g., buffer free of any agonist). In addition to resensitizing ion channels and receptors, this delivery of buffer onto cells between ligand and drug exposure serves to wash out ligands and drugs previously administered to the cell. Thus, in this aspect, the system is used to screen for an agonist or modulator or drug of a specific ion-channel by providing a periodically responsive ion channel sensor. For example, by providing pulsed or steady-state flow delivery of buffer to the sensor, the system provides a cell that is resensitized when exposed to a channel outlet delivering a candidate agonist or modulator or drug.

To obtain desired data, variable exposure rates of cell(s) to individual streams of sample and buffer and variable pressure drops across the sensor chamber can be implemented by the system, either from pre-programmed instructions or in response to feed-back signals from a detector in electrical communication with the patch clamp electrode (e.g., based on a detected signal or in real-time) or in optical communication in the case of fluorescence read out of GPCRs activity.

The system thus can be used to change microenviroments rapidly around a cell comprising a receptor/ion-channel. For example, the system can provide a periodically responsive ion channel. Because of the small dimensions of the substrates and microchannels used herein, which allows for rapid mass transport, the system enables a user to screen for drugs, in some instances, at the rate of hundreds per second (e.g., millions per hour) using one patch clamp sensor, provided drugs and resensitization solutions are delivered sequentially at a comparable rate to the sensor. As discussed above, exposing rates can be modified to account for the physiological responses of a cell-based sensor, e.g., providing slower exposing rates for receptors that equilibrate slowly. The system also allows for redundancy and multiplicity, that is the exposure of multiple sensors to the same conditions at the same time, which is advantageous.

Dose-response curves provide valuable information regarding the actions and potencies of drugs. Obtaining dose-response curves using traditional methods involving micropipettes often can be time consuming and tedious. The present invention, which uses microfluidics for the rapid and controlled manipulation of the microenvironment around cell(s), is uniquely suited for dose-response measurements. Dose-response relationships most often follow a sigmoidal curve in a lin-log plot, and can be described by the Hill logistic functions:

I=Iₘₐₓ/[1+(EC₅₀/C)ⁿ]

Where I is the whole-cell current, C is the concentration of ligands, Iₘₐₓ is the maximal current (e.g., when all channels are in the open state), EC₅₀ is the half-maximal value (e.g., when half of the receptor population is activated, and often equals K_{D}, the dissociation constant of the ligand), and n is the Hill coefficient that reflects the stoichiometry of ligand binding to the receptor. One of skill in the art, having the benefit of this disclosure would understand how to use the system to generate a dose response curve and to detect and characterize agonist and antagonists. See for example, U.S. patent application Ser. Nos. 10/345,107 and 10/645,834, which are hereby incorporated by reference in their entirety.

The ability of a drug molecule to activate a receptor-mediated response is a graded property, rather than an all-or-nothing property. If a series of chemically related agonists acting on the same receptor are tested on a cell, the maximal response (e.g., the largest response that can be produced by an agonist in high concentration) generally differs from one agonist to another. Some compounds (known as “full agonists”) can produce a maximal response whereas others, referred to “partial agonists”, can only produce a submaximal response. An “partial agonist” can therefore act as a “weak antagonist” by hampering a full agonist from binding a receptor. Thus, by using a defined ion-channel together with a known agonist that produces a maximal response, the grade of an agonist's activity can be monitored.

In one aspect, the system is used to screen for antagonists of ion-channel activity. Suitable ion-channels which can be evaluated include: (i) ion channels that do not de-sensitize; (ii) ion-channels that desensitize (iii) ion-channels that desensitize but which mediate large current fluctuations when activated; and (iv) ion-channels whose desensitizing property is blocked by irreversible binding of an allosteric modulator (e.g., such as a lectin). To detect antagonists, the ion-channels or receptors expressed by a biosensor need to be activated or “tested” by an agonist during, before, or after; application of the antagonist. For example, different antagonists can be applied together with a well-defined agonist with known pharmacological properties. Antagonists at different concentrations also can be loaded into microchannels together with agonists at a constant concentration.

### EXAMPLES

The invention will now be further illustrated with reference to the following examples. It will be appreciated that what follows is by way of example only and that modifications to detail may be made while still falling within the scope of the invention.

### Example 1

### Microfabrication of a Substrate

FIG. 2B-D show examples of microchannels fabricated in polydimethylsiloxane (PDMS), which was replicated from a silicon master made by deep reactive ion etching in SF₆. Masks for photolithography were produced using standard e-beam writing on a JEOL JBX-5DII electron beam lithography system (medium reflective 4″ chrome masks and Shipley UV5 resists, 50 keV acc. voltage, dose 15 μC/cm⁻², exposure current 5 nA). The resist was spin coated at 2000 rμm for 60 s giving 250 nm of resist and soft baked for 10 minutes at 130° C. on a hotplate before exposure. The pattern was post exposure baked for 20 minutes in an oven at 130° C. and developed for 60 s in Shipley MF24-A, rinsed in DI water and etched in a reactive ion etcher (Plasmatherm RIE m-95, 30 s, 50 W, 250 mTorr, 10 ccm O₂). The chrome was etched for 1-2 minutes in Balzers chrome etch #4, the mask was stripped of the remaining resist using Shipley 1165 remover and rinsed in acetone, isopropanol and DI water. A 3″, [100], two sides polished, low N-doped Silicon wafers with 700 nm of thermally grown silicon dioxide and a total thickness of 380 μm was cleaned in a reactive ion etcher Plasmatherm RIE m-95 (30 s, 50 W, 250 mTorr, 10 ccm O₂), spin coated with Shipley S-1813 photoresist at 4000 rμm, giving 1.3 μm of resist.

110 mJ/cm⁻² at 400 nm wavelength on a Carl Süss MA6 mask aligner. The wafer was developed for 45 s in Shipley MF319 rinsed in DI water and ashed in a reactive ion etcher (Plasmatherm RIE m-95, 30 s, 50 W, 250 mTorr, 10 ccm O₂). The wafer was hard baked for 10 minutes at 130° C., the silicon dioxide was etched with SioTech buffered oxide etch and rinsed in DI water. The wafer was stripped of the remaining resist with acetone, rinsed in isopropanol and DI water. The other side of the wafer was spin coated with Shipley AZ4562 photoresist at 3000 rμm for 30 seconds giving approximately 8 μm of resist, soft baked for 3 minutes at 100° C. on a hotplate and exposed for a dose of 480 mJ/cm⁻² at 400 nm wavelength on a Carl Süss MA6 mask aligner. The pattern was developed for 200 seconds in Shipley MF312 and DI water in 50:50 mix, rinsed in DI water, and ashed in a reactive ion etcher (Plasmatherm RIE m-95, 30 seconds, 50 W, 250 mTorr, 10 ccm O₂).

The pattern defined in the photoresist AZ4562, the recording chamber and the combined access holes and sample wells was etched in a STS Multiplex deep reactive ion etcher using SF₆ as etching gas and C₄F₈ as passivation gas at 600 W of RF power and 30 W of platen power. The system was operating at a constant APC angle of 74% and the etching time was 12 seconds with an overrun time of 1 second, and the passivation time 8 seconds with an overrun time of 1 second. The etching rate was approximately 4.9 μm/minute and the etching time 60 minutes resulting in a depth of approximately 300 μm. The wafer was stripped of the remaining resist in acetone, rinsed in isopropanol and DI water.

The pattern in silicon dioxide defining the microchannels was etched with the same system as before but with 800 W of RF power, at a constant APC angle of 68% and the etching time was 7 s with an overrun time of 0.5 s, and the passivation time 4 second with an overrun time of 1 second. The etching rate was approximately 3.3 μm/min and the etching time 30 minutes resulting in a depth of 100 μm. The wells and the recording chamber were completely etched through resulting in holes in the wafer at these points. The channels were sealed to a 3″, 1000 μm thick wafer of Corning #7740 borosilicate glass using anodic bonding at a temperature of 450° C. and a voltage of 1000 V. The maximum current during bonding was typically 500 μA.

### Example 2

### Microfluidic Switching

### The Microsystem

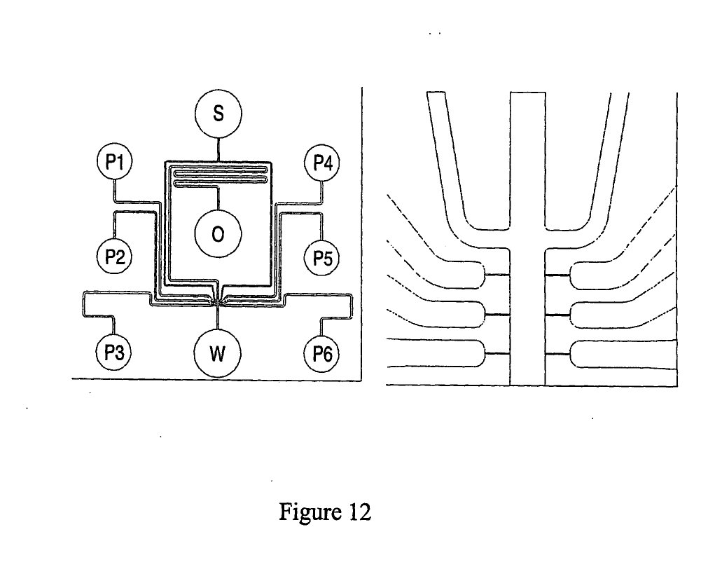

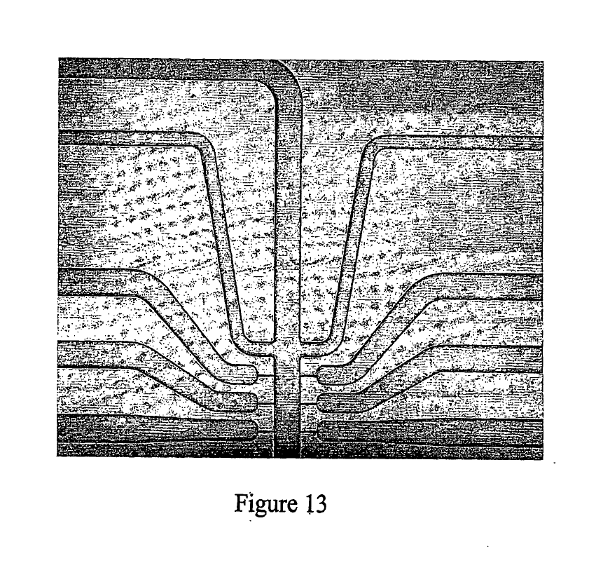

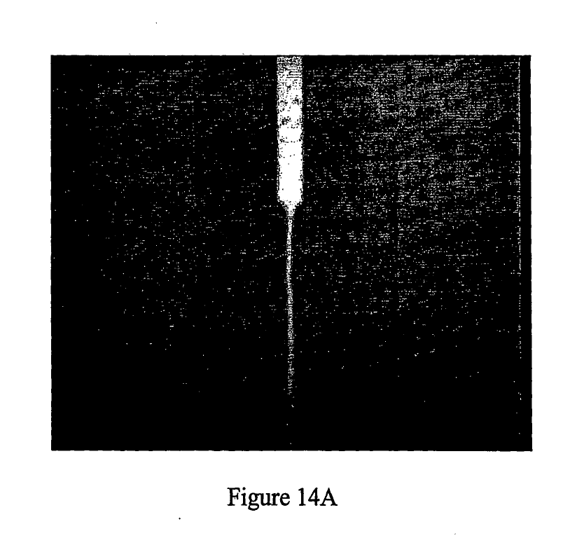

FIGS. 12A-B and 13 shows an example of a microfluidic system where rapid switching occurs in a closed sensor chamber. The chip is made from an injection molded PDMS slab containing microchannels which is plasma-bonded to a glass substrate allowing observation and fluorescence readout in an inverted microscope.

In this sensor chamber there are six openings for cell holding, three on each side. These openings connect to microchannels leading to wells P (patch) 1-6 intended for communicating pressure for cell holding and capturing, and containing silver/silver chloride working electrodes for patch-clamp recordings. Just at the opening a 50 μm long 2 μm×2 μM channel protrudes, the small dimensions allowing a high resistance electrical seal as well as the possibility to hold a cell without sucking it through the channel. The channel length is optimized to form a good electrical seal without having a too high fluidic or electrical access resistance. After the 50 μm section, the channel widens to a 50 μm wide and 30 μm high channel which fans out and soon reaches 70 μm in width to minimize resistances. All six channels are matched in length to avoid differences in the measurement situation at the different patch sites. The patch wells can be filled with up to 5 μl of buffer.

On the top of the recording chamber three microchannels for substance or buffer delivery converges. The single delivery channel is 30 μm high and 70 μm wide and leads to the open well via a meandering pattern to match the flow resistance to that of the buffer channels. This well is open to make it possible to deliver different solutions to the sensor chamber: first the living cells, and then a molecule such as a receptor ligand perhaps in stepwise increasing concentrations to perform dose-response measurements. Since the main flow of the system is driven by a negative pressure through the waste the contents of the open well can be exchanged by pipetting while the system still is running.

The buffer channels are 35 μm wide and 30 μm high and converge to a single 70 μm wide channel just before the switching/buffer well. This well not only contains buffer, but also is the connection point for the switching pressure source, which actuates the rapid fluidic switch at the cell positions by a change from atmospheric pressure to a balanced negative pressure in the 0.5-10 kPa range.

At the bottom of the sensor chamber a single 70 μm wide and 30 μm high waste channel leads to the W (waste) well which contains a silver/silver chloride ground electrode and a pressure connection for external driving pressure. The open, switching and waste wells can contain up to 15 μl.

### Setup

The entire system is used in an inverted microscope for visual confirmation of the cell capture and possibility to record fluorescence data in addition to electrical patch-clamp measurement.

As a pressure source for the driving and switching pressures, small aquarium pumps connected to dampening dead-volumes are used. Each pump is leaking through a pressure resistance and a proportional valve and between these a differential pressure sensor is measuring the pressure compared to atmosphere. This is the controlled pressure used in the microsystem. A computer-controller feedback loop continually adjusts the proportional valve to achieve the correct pressure.

An external lid, sealed to the chip by double adhesive tape is used to connect three pressure sources and seven electrodes. The pressure sources are connected through Upchurch Vacutight® fittings and the electrodes are made from 1.0 mm diameter 99.95% pure silver rods (Goodfellow) that are pressed through the lid in tight holes to achieve pressure seal. The electrode tips were chlorinated after mounting, using a 9V battery and 0.1M HCl.

For driving pressure a negative/vacuum pressure of 0.5 to 10 kPa is used at the waste well. For the cell catching and holding a 10 ml syringe is used for suction. For switching, an electrically actuated three-way pneumatic switch was used to switch, between atmospheric pressure and a negative pressure between 0.5 and 10 kPa.

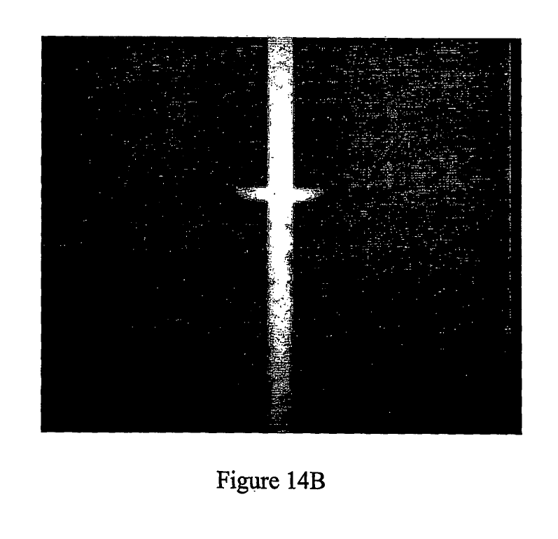

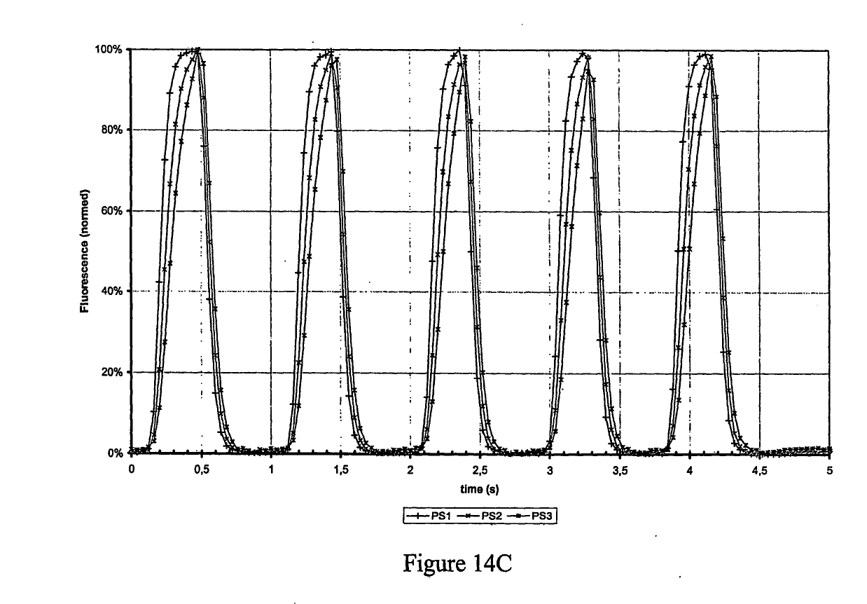

When atmospheric pressure is applied to the S (switch/buffer) well, a triple laminar flow is formed in the sensor chamber (see FIG. 14 A) Since the flow resistance in the buffer channels is similar to the channel leading to the open well, the negative pressure on the waste well draws equal flows from the three channels. Since the Reynolds number in the microfluidic system is low, <0.5, those flows will stay parallel to each other and not mix otherwise than by diffusion.

When the pressure source at the S well is switched to a negative pressure slightly larger than the driving pressure on the W well, the flow in the buffer channel will change direction. Therefore a part of the flow from the open 0 well will be drawn towards the S well. This motion causes a rapid switch of the fluid surrounding the cells in the sensor chamber, from buffer to the content of the open well. (See FIG. 14B)

### Experiment

To begin the experiment, the P wells were filled with intracellular buffer. Then the S well was filled with extracellular buffer before the system was sealed with the lid and placed in the setup. Immediately before the flow was started cells in suspension were pipetted into the open well. A vacuum pressure of 0.5 kPa was applied to the W well, giving a flow speed just enough to keep the cells from settling on the channel floor. Applying a switching pressure of 0.6 kPa switched the cells out to the sensor chamber walls. When negative pressure was applied to the P wells with a syringe, a portion of the cell suspension flow was drawn into the 2×2 μm openings, cathing one cell or cell cluster at each patch site.

After catching cells in at least five of the six positions, was turned off to rinse the cells with buffer while the cell solution in the open well was replaced with the first sample. For the first experiment, shown in FIG. 14, the sample was a solution of 20 μM fluorescein in extracellular buffer. For sample switching the switch time should be minimized, and therefore a much higher flow-speed than for cell capture was used. The driving pressure was set to −8.0 kPa and a switching pressure of −8.6 kPa was used.

This experiment confirmed the stability of the fluidic switch even when the sensor chamber was partly blocked by cells at the sensor sites and also gave a measurement of the switch-time by time-lapse frame capturing and numerical treatment of the fluorescence data at the surface of the cells. In FIG. 14A you can see the triple laminar flow where fluorescein is sheathed by buffer flows and FIG. 14B where the fluorescein is switched to flush the cells. These two states also represent the nonmed 0% fluorescence respective 100% fluorescence of the graphs in FIG. 14C with maximal fluorescein concentration around all cells at the sensor sites.

As seen in FIG. 14C, the 10-90% risetime at the patch-site nearest the inflows is around 120 ms and longer at the more downstreams sites. However, this is a high overestimation of the fluidic switch around the cells, because the fluorescence signal contains a lot of out-of-focus signal from fluorophore in the channel above the cell. It can also be noted that since the flow speed of the system is quite high, diffusional broadening of the concentration front is very small, compared to the difference in front arrival due to the parabolic flow profile in laminar fluidics.

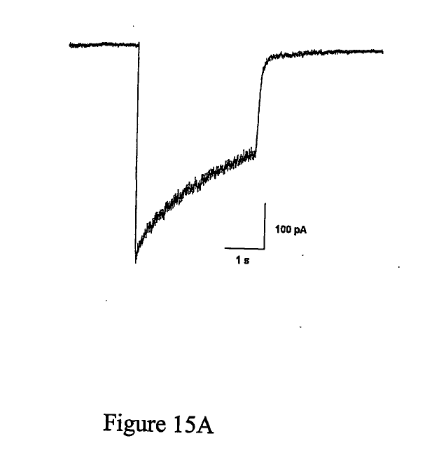

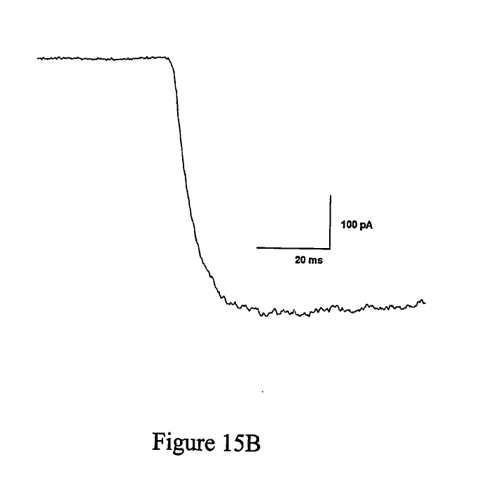

As a second experiment, a dose-response for the GABAA ion channel stimulated with GABA was recorded. GABA was used in stepwise increasing concentrations, giving a complete dose-response profile for GABA on single cells. Between each concentration, the cells were rinsed with buffer until the baseline current was restored, and the open wells were refilled with the next higher concentration. In FIG. 15 it is shown that the ion channel current read from a patch-clamped cell in the first sensor position using a GABA concentration of 1 mM. The 10-90% risetime here is shorter than 20 ms.

In FIG. 15A, the switching time is not as fast when switching back to buffer. This is because of the analyte drawn up the buffer channel, where it is subject to diffusion over a long time, making the cells pass through a large diffused zone before being reached by the pure buffer.

What is claimed is:

1. A method for modulating, controlling, preparing, or studying receptors, comprising:
a) providing a substrate, the substrate comprising:
a sensor channel comprising a plurality of sensor positioning channels, and a delivery channel configured to deliver one or more of an agent, agonist, or antagonist to the sensor chamber; and
(b) sequentially exposing a biosensor to different fluid streams optionally comprising an agent, wherein sensors are associated with one or more sensor positioning channels.

2. The method of claim 1, wherein the sensor is a cell or a vesicle.

3. The method of claim 1, wherein the sensor positioning channel is a patch channel.

4. The method of claim 1, wherein sequentially exposing comprises solution exchange around a sensor.

5. The method of claim 4, wherein the solution exchange is pressure driven.

6. The method of claim 1, wherein the substrate further comprises one or more of at least one pressure source adapted to provide positive and negative pressure to the sensor channel and the plurality of sensor positioning channels; a buffer reservoir in fluid communication to the sensor channel; an inlet reservoir in fluid communication with the sensor channel; an inlet reservoir in fluid communication with the sensor channel; or a waste reservoir in fluid communication with the sensor channel.

7. The method of claim 6, further comprising applying negative pressure from the waste reservoir.

8. The method of claim 6, further comprising applying positive pressure to the buffer reservoir.

9. The method of claim 6, further comprising applying negative pressure on the plurality of sensor channels.

10. The method of claim 1, wherein the openings of the sensor positioning channels comprise protruded surfaces.

11. The method of claim 10, wherein the protruded surface defining an opening comprises one or more of a microchannel, a column, a pyramidal element, rod or reeve.

12. The method of claim 1, wherein electrical resistance between a sensor and the system comprises at least about 100 Mohm.

13. The method of claim 6, wherein the method further comprises measuring electrical properties of the cell.

14. The method of claim 1, wherein the sensor chamber comprises a buffer, at least one agonist, at least one antagonist, at least one sample, or a combination thereof.

15. The method of claim 1, wherein the exposing is selectively exposing the biosensor to a selected concentration of sample.

16. The method of claim 1, wherein the exposing is selectively for a selected time.

17. The method of claim 1, further comprising providing to the sensor positioning channels one or more buffers.

18. The method of claim 1, further comprising exposing the biosensor to the one or more buffers.

19. The method of claim 1, wherein the exposing the biosensor to one or more buffers is interspersed between the exposing to one or more samples.

20. The method of claim 1, wherein the exposing to one or more buffers is a wash period.

21. The method of claim 1, wherein the exposing to one or more buffers is a rest period.

22. The method of claim 1, wherein the exposing to one or more buffers is a wash and a rest period.

23. The method of claim 1, wherein a rest period in buffer is between a series of sample exposures and interdigitated by one or more wash periods in buffer.

24. The method of claim 1, wherein the receptors are exposed to ligand solutions in order of increasing concentrations

25. The method of claim 1, wherein the receptors are exposed to ligand solutions in order of decreasing concentrations

26. The method of claim 1, wherein the agent is a candidate drug; a known drug; a suspected carcinogen; a known carcinogen; a candidate toxic agent, a known toxic agent; and an agent that acts directly or indirectly on ion channels.

27. The method of claim 1, wherein the method is method for studying the memory properties of a receptor.

28. The method of claim 27, wherein the memory functions are short-term, medium-term, or long-term memory functions.

29. The method of claim 27, wherein effects of an agent on memory properties of a biosensor are studied.

30. The method of claim 1, wherein the exposing further comprises producing pressure drops across one or more channels.

31. The method of claim 1, wherein the cell-based biosensor comprises a patch-clamped cell or patch-clamped cell membrane fraction.

32. The method of claim 1, wherein the cell-based biosensor comprises an ion-channel.

33. The method of claim 32, wherein the ion-channel is a G-Protein Coupled Receptor.

34. A system comprising:
a substantially planar substrate in communication with at least one conducting element, wherein the substantially planar structure comprises a sensor channel comprising a plurality of sensor positioning channels.

35. The system of claim 34, wherein the sensor positioning channels comprise electrode channels.

36. The system of claim 34, further comprising at least one pressure source adapted to provide positive and negative pressure to the sensor channel and the plurality of sensor positioning channels.

37. The system of claim 34, further comprising a buffer reservoir in fluid communication to the sensor channel.

38. The system of claim 34, further comprising an inlet reservoir in fluid communication with the sensor channel.

39. The system of claim 34, further comprising a waste reservoir in fluid communication with the sensor channel.

40. The system of claim 34, wherein further comprising a mechanism for providing fluid flow for establishing and maintaining an electrically resistant seal between a cell and a conducting element.

41. The system of claim 34, wherein the openings of the sensor positioning channels comprise protruded surfaces.

42. The system of claim 41, wherein the protruded surface defining an opening comprises one or more of a microchannel, a column, a pyramidal element, rod or reeve.

43. The system of claim 34, wherein electrical resistance between a sensor and the system comprises at least about 100 Mohm.

44. The system of claim 34, wherein the system is used for one or more of patch clamping measuring a parameter of a sensor.

45. The system of claim 44, wherein the parameter measured using fluorescence.

46. The system of claim 44, wherein the parameter is one of more of an ion channel activity, currents across sensor membranes, voltage across the membranes, or capacitance across the membranes.

47. A system for rapid switching, comprising:
a substantially planar substrate in communication with at least one conducting element, wherein the substantially planar structure comprises:
a sensor chamber comprising a plurality of sensor positioning channels,
a delivery channel, at least one buffer/agent delivery channel in communication with the sensor chamber,
a waste channel in communication with the sensor chamber,
a buffer well,
a negative pressure source communicated through the waste channel, and
a switching pressure source communicated through the buffer well, and a ground electrode.

48. The system of claim 47, wherein the buffer/agent delivery channels are from between about 25 to about 45 um wide and about 15 to about 45 um high and converge to a single channel that is from between about 55 to about 85 um wide.

49. The system of claim 47, wherein the buffer/agent delivery channels are about 35 um wide and about 30 um high and converge to a single channel that is about 70 um wide.

50. The system of claim 47, wherein the sensor chamber is from between about 50 to about 100 um wide and from between about 15 to about 45 um high.

51. The system of claim 47, wherein the sensor chamber is about 70 um wide and about 30 um high.

52. The system of claim 47, wherein the delivery channel is from between about 50 to about 100 um wide and from between about 15 to about 45 um high.

53. The system of claim 47, wherein the delivery channel is bout 70 um wide and about 30 um high.

54. The system of claim 47, wherein the sensor positioning channels comprise openings into the sensor chamber and wherein the opening are from between about 50 um long.

55. The system of claim 47, wherein the sensor positioning channels after between about a 25to about a 75 um section widens to between about 25 to about 75 um wide and between about 15 to about 45 um high.

56. The system of claim 47, wherein the sensor positioning channels after about a 50 um section widen to between about 50 um wide and about 30 um high.

57. The system of claim 47, wherein the buffer well comprises a volume of between about 5 uL and about 30 uL.

58. The system of claim 47, further comprising a waste well in communication with the waste channel.

59. The system of claim 47, wherein the waste well comprises a volume of between about 5 uL and about 30 uL.

60. The system of claim 47, wherein the conducting element comprise electrodes.

61. The system of claim 47, wherein the ground electrode is contained within the waste chamber.

62. The system of claim 47, wherein the sensor positioning channels are in communication with wells for communicating pressure.

63. The system of claim 47, wherein the sensor positioning channels are the same length, wherein the sensor positioning channels comprise electrodes.

64. A method for modulating, controlling, preparing, or studying receptors, comprising:
providing a microfluidic system, wherein the microfluidic system comprises a substrate in communication with at least one conducting element, wherein the substantially planar structure comprises:
a sensor chamber comprising a plurality of sensor positioning channels,
a delivery channel, at least one buffer/agent delivery channel in communication with the sensor chamber,
a waste channel in communication with the sensor chamber,
a buffer well,
a negative pressure source communicated through the waste channel, and a switching pressure source communicated through the buffer well, and a ground electrode;
capturing a biosensor at an opening of a sensor positioning channel; and
exposing the biosensor to an agent.

65. The method of claim 64, wherein the method further comprising exposing the biosensor to a buffer, wherein the switching between buffer and agent is rapid.

66. The method of claim 65, wherein rapid comprises between about 10 μs and about 100 seconds.

67. The method of claim 66, wherein switching between buffer and agent comprises a switching pressure of between about −7.6 and about −9.6 kPa.

68. The method of claim 64, wherein a capture pressure is applied to the system and comprises from between about 0.4 to about 0.8 kPa.

69. The method of claim 64, wherein a driving pressure is applied to the system after a biosensor is captured and comprises from between about −7 to about −9 kPa.

70. The method of claim 65, wherein switching between buffer and agent is done one or more times.

71. The method of claim 70, wherein switching between buffer and agent is done at a rate of five time in about 4.5 seconds.

72. The method of claim 65, wherein switching between buffer and agent comprises a fluidic switch time.

73. The method of claim 72, wherein the fluidic switch time comprises from between about 15 to about 35 ms.
